SEQUENCE LISTING (112 sequences)

SEQ ID NO: 1 - DNA, 63 bp, Artificial Sequence
  synthetic construct
  source (1)..(63)
atggctctgcctgtgaccgctctgctgctgcctctggctctgctgctgcacgccgcaagacct

SEQ ID NO: 2 - DNA, 726 bp, Artificial Sequence
  synthetic construct
  source (1)..(726): GPC3 scFv
gatgtcgtgatgacccagtccccactgtccctgccagtgacaccaggagagcctgcatccatctcttgccggagctcccagtctctggtgcacagcaacggcaatacctacctgcactggtatctgcagaagccaggccagagcccccagctgctgatctacaaggtgtccaaccggttctctggagtgccagaccggttcagcggctccggctctggcaccgatttcacactgaagatcagcagggtggaggcagaggacgtgggcgtgtactattgctcccagaatacccacgtgccccctacatttggccagggcaccaagctggagatcaagggaggaggaggcagcggcggaggaggctccggcggcggcggctctcaggtgcagctggtgcagtccggagcagaggtgaagaagcctggagccagcgtgaaggtgtcctgtaaggcctctggctacaccttcacagattatgagatgcactgggtgcggcaggcacctggacagggactggagtggatgggcgccctggacccaaagaccggcgatacagcctactctcagaagtttaagggcagggtgaccctgacagccgacgagagcacctccacagcctatatggagctgtctagcctgcgcagcgaggataccgccgtgtactattgcacccgcttctacagttacacttattgggggcagggcactctggtcacagtctcttca

SEQ ID NO: 3 - DNA, 726 bp, Artificial Sequence
  synthetic construct
  source (1)..(726): CD19 scFv
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 4 - DNA, 738 bp, Artificial Sequence
  synthetic construct
  source (1)..(738): BCMA scFv
gacatcgtgctgacccagagccccgcttctttagctgtgtctttaggagagagggccaccatcaattgtcgtgcctccgagtccgtgtccgtgattggcgcccatttaatccactggtaccagcagaaacccggccagccccccaagctgctgatctatttagccagcaatctggagaccggcgtgcccgccagatttagcggaagcggctccggcaccgattttactttaaccatttcctctttacaagctgaggacgccgccatctactactgtttacagagcagaatctttcctcgtaccttcggccaaggtaccaagctggaaatcaagggatccaccagcggctccggaaagcccggttccggagagggcagcacaaagggccaagttcagctcgtgcagagcggctccgagctgaagaagcccggcgcttccgtgaaggtgagctgcaaggccagcggatacaccttcaccgactactccatcaactgggttcgtcaagctcccggtcaaggtctggagtggatgggctggatcaacaccgaaaccagagagcccgcctatgcctacgacttcagaggtcgtttcgtgttctctttagacacaagcgtgagcaccgcctatttacagatcagctctttaaaggccgaggataccgccgtgtactactgcgctcgtgactacagctacgccatggactactggggccaaggtacactggtgaccgtgtccagc

SEQ ID NO: 5 - DNA, 777 bp, Artificial Sequence
  synthetic construct
  source (1)..(777): MSLN scFv
caggtgcagctgcaacagagcggccctggcctggtgacaccttcccagaccctcagcctcacctgcgccatctccggcgattccgtgtccagcaatagcgccacctggaactggatcagacagagccctagcagaggcctggagtggctgggcaggacctattataggagcaagtggtacaacgactacgccgtgtccgtcaagtccagaatgagcatcaaccccgacaccagcaagaatcagttcagcctgcagctgaacagcgtgacccccgaggacacagccgtgtattactgcgccaggggcatgatgacctactactacggcatggacgtgtggggccaaggcaccacagtgaccgtgagcagcggcattctgggcagcggaggaggcggatccggcggcggaggcagcggaggaggaggcagccagcctgtgctgacccagtccagctccctgtccgcttcccctggagcctccgcttccctgacctgcaccctgaggtccggcatcaatgtgggcccctacaggatctactggtatcagcagaagcctggcagccccccccagtacctgctgaactacaagagcgacagcgacaagcagcagggcagcggagtgcctagcagattcagcggatccaaggacgcctccgccaatgctggcgtgctgctgatctccggcctgagaagcgaggacgaagccgactactactgcatgatctggcacagcagcgctgccgtgtttggaggcggcacccaactgacagtcctgtcc

SEQ ID NO: 6 - DNA, 723 bp, Artificial Sequence
  synthetic construct
  source (1)..(723): HER2 scFv
caggtccagctgcagcagagcggacccgagctcaagaagcccggcgagaccgtgaagatcagctgtaaggccagcggctacccctttaccaactatggaatgaactgggtgaagcaagctcccggccagggcctgaagtggatgggctggatcaacacaagcaccggagaaagcaccttcgctgacgacttcaagggcagatttgacttctccctcgagacctccgccaacaccgcctacctgcagatcaacaatctgaagtccgaggacagcgctacctacttctgcgccagatgggaggtgtaccatggctacgtcccttattggggccaaggcaccaccgtgaccgtgagcagcggaggaggcggttctggaggcggcggcagcggcggcggcggcagcgacattcagctgacccagtcccacaagttcctgagcacctccgtgggcgacagggtgagcattacctgcaaggccagccaggacgtgtacaatgccgtggcctggtaccagcagaagcctggacagagccccaagctgctgatctacagcgcttcctccaggtacaccggcgtcccttccagatttaccggctccggctccggccccgacttcaccttcaccatcagctccgtgcaggccgaagacctggccgtgtacttctgccaacagcacttcaggacccccttcacctttggcagcggaaccaagctggagatcaaa

SEQ ID NO: 7 - DNA, 138 bp, Artificial Sequence
  synthetic construct
  source (1)..(138): CD8
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatc

SEQ ID NO: 8 - DNA, 69 bp, Artificial Sequence
  synthetic construct
  source (1)..(69): CD8
tacatctgggcgcccttggccgggacttgtggggtccttctcctgtcactggttatcaccctttactgc

SEQ ID NO: 9 - DNA, 123 bp, Artificial Sequence
  synthetic construct
  source (1)..(123): CD28
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 10 - DNA, 126 bp, Artificial Sequence
  synthetic construct
  source (1)..(126): 4-1BB41BB
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 11 - DNA, 339 bp, Artificial Sequence
  synthetic construct
  source (1)..(339): CD3CD3zeta
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 12 - DNA, 54 bp, Artificial Sequence
  synthetic construct
  source (1)..(54): T2A
gagggcagaggaagtcttctaacatgcggtgacgtggaggagaatcccggccct

SEQ ID NO: 13 - DNA, 57 bp, Artificial Sequence
  synthetic construct
  source (1)..(57): P2A
gccaccaattttagcctgctgaaacaggccggagacgtggaggaaaaccccggcccc

SEQ ID NO: 14 - DNA, 578 bp, Artificial Sequence
  synthetic construct
  source (1)..(578): IRES
tccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccaca

SEQ ID NO: 15 - DNA, 1106 bp, Artificial Sequence
  synthetic construct
  source (1)..(1106): S1
acggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaggcggaggatcaggcggaggctcccagtatctgtttttgccaccaaatcgttacattttccatggcgctgaggtatattcagactctgaagatgacgtctga

SEQ ID NO: 16 - DNA, 1167 bp, Artificial Sequence
  synthetic construct
  source (1)..(1167): S2
atggcagagccagacccctctcaccctctggagacccaggcagggaaggtgcaggaggctcaggactcagattcagactctgagggaggagccgctggtggagaagcagacatggacttcctgcggaacttattctcccagacgctcagcctgggcagccagaaggagcgtctgctggacgagctgaccttggaaggggtggcccggtacatgcagagcgaacgctgtcgcagagtcatctgtttggtgggagctggaatctccacatccgcaggcatccccgactttcgctctccatccaccggcctctatgacaacctagagaagtaccatcttccctacccagaggccatctttgagatcagctatttcaagaaacatccggaacccttcttcgccctcgccaaggaactctatcctgggcagttcaagccaaccatctgtcactacttcatgcgcctgctgaaggacaaggggctactcctgcgctgctacacgcagaacatagataccctggagcgaatagccgggctggaacaggaggacttggtggaggcgcacggcaccttctacacatcacactgcgtcagcgccagctgccggcacgaatacccgctaagctggatgaaagagaagatcttctctgaggtgacgcccaagtgtgaagactgtcagagcctggtgaagcctgatatcgtcttttttggtgagagcctcccagcgcgtttcttctcctgtatgcagtcagacttcctgaaggtggacctcctcctggtcatgggtacctccttgcaggtgcagccctttgcctccctcatcagcaaggcacccctctccacccctcgcctgctcatcaacaaggagaaagctggccagtcggaccctttcctggggatgattatgggcctcggaggaggcatggactttgactccaagaaggcctacagggacgtggcctggctgggtgaatgcgaccagggctgcctggcccttgctgagctccttggatggaagaaggagctggaggaccttgtccggagggagcacgccagcatagatgcccagtcgggggcgggggtccccaaccccagcacttcagcttcccccaagaagtccccgccacctgccaaggacgaggccaggacaacagagagggagaaaccccag

SEQ ID NO: 17 - DNA, 771 bp, Artificial Sequence
  synthetic construct
  source (1)..(771): S3
atggtgggggccggcatcagcacacccagtggcattccagacttcagatcgccggggagtggcctgtacagcaacctccagcagtacgatctcccgtaccccgaggccatttttgaactcccattcttctttcacaaccccaagccctttttcactttggccaaggagctgtaccctggaaactacaagcccaacgtcactcactactttctccggctgcttcatgacaaggggctgcttctgcggctctacacgcagaacatcgatgggcttgagagagtgtcgggcatccctgcctcaaagctggttgaagctcatggaacctttgcctctgccacctgcacagtctgccaaagacccttcccaggggaggacattcgggctgacgtgatggcagacagggttccccgctgcccggtctgcaccggcgttgtgaagcccgacattgtgttctttggggagccgctgccccagaggttcttgctgcatgtggttgatttccccatggcagatctgctgctcatccttgggacctccctggaggtggagccttttgccagcttgaccgaggccgtgcggagctcagttccccgactgctcatcaaccgggacttggtggggcccttggcttggcatcctcgcagcagggacgtggcccagctgggggacgtggttcacggcgtggaaagcctagtggagcttctgggctggacagaagagatgcgggaccttgtgcagcgggaaactgggaagcttgatggaccagacaaa

SEQ ID NO: 18 - DNA, 945 bp, Artificial Sequence
  synthetic construct
  source (1)..(945): S4
atgaagatgagctttgcgttgactttcaggtcagcaaaaggccgttggatcgcaaaccccagccagccgtgctcgaaagcctccattgggttatttgtgccagcaagtcctcctctggaccctgagaaggtcaaagagttacagcgcttcatcaccctttccaagagactccttgtgatgactggggcaggaatctccaccgaatcggggataccagactacaggtcagaaaaagtggggctttatgcccgcactgaccgcaggcccatccagcatggtgattttgtccggagtgccccaatccgccagcggtactgggcgagaaacttcgtaggctggcctcaattctcctcccaccagcctaaccctgcacactgggctttgagcacctgggagaaactcggaaagctgtactggttggtgacccaaaatgtggatgctttgcacaccaaggcggggagtcggcgcctgacagagctccacggatgcatggacagggtcctgtgcttggattgtggggaacagactccccggggggtgctgcaagagcgtttccaagtcctgaaccccacctggagtgctgaggcccatggcctggctcctgatggtgacgtctttctctcagaggagcaagtccggagctttcaggtcccaacctgcgttcaatgtggaggccatctgaaaccagatgtcgttttcttcggggacacagtgaaccctgacaaggttgattttgtgcacaagcgtgtaaaagaagccgactccctcttggtggtgggatcatccttgcaggtatactctggttacaggtttatcctcactgcctgggagaagaagctcccgattgcaatactgaacattgggcccacacggtcggatgacttggcgtgtctgaaactgaattctcgttgtggagagttgctgcctttgatagacccatgctga

SEQ ID NO: 19 - DNA, 933 bp, Artificial Sequence
  synthetic construct
  source (1)..(933): S5
atgcgacctctccagattgtcccaagtcgattgatttcccagctatattgtggcctgaagcctccagcgtccacacgaaaccagatttgcctgaaaatggctcggccaagttcaagtatggcagattttcgaaagttttttgcaaaagcaaagcacatagtcatcatctcaggagctggtgttagtgcagaaagtggtgttccgaccttcagaggagctggaggttattggagaaaatggcaagcccaggacctggcgactcccctggcctttgcccacaacccgtcccgggtgtgggagttctaccactaccggcgggaggtcatggggagcaaggagcccaacgccgggcaccgcgccatagccgagtgtgagacccggctgggcaagcagggccggcgagtcgtggtcatcacccagaacatcgatgagctgcaccgcaaggctggcaccaagaaccttctggagatccatggtagcttatttaaaactcgatgtacctcttgtggagttgtggctgagaattacaagagtccaatttgtccagctttatcaggaaaaggtgctccagaacctggaactcaagatgccagcatcccagttgagaaacttccccggtgtgaagaggcaggctgcgggggcttgctgcgacctcacgtcgtgtggtttggagaaaacctggatcctgccattctggaggaggttgacagagagctcgcccactgtgatttatgtctagtggtgggcacttcctctgtggtgtacccagcagccatgtttgccccccaggtggctgccaggggcgtgccagtggctgaatttaacacggagaccaccccagctacgaacagattcaggtttcatttccagggaccctgtggaacgactcttcctgaagcccttgcctgtcatgaaaatgaaactgtttcttaa

SEQ ID NO: 20 - DNA, 1065 bp, Artificial Sequence
  synthetic construct
  source (1)..(1065): S6
atgtcggtgaattacgcggcggggctgtcgccgtacgcggacaagggcaagtgcggcctcccggagatcttcgaccccccggaggagctggagcggaaggtgtgggaactggcgaggctggtctggcagtcttccagtgtggtgttccacacgggtgccggcatcagcactgcctctggcatccccgacttcaggggtccccacggagtctggaccatggaggagcgaggtctggcccccaagttcgacaccacctttgagagcgcgcggcccacgcagacccacatggcgctggtgcagctggagcgcgtgggcctcctccgcttcctggtcagccagaacgtggacgggctccatgtgcgctcaggcttccccagggacaaactggcagagctccacgggaacatgtttgtggaagaatgtgccaagtgtaagacgcagtacgtccgagacacagtcgtgggcaccatgggcctgaaggccacgggccggctctgcaccgtggctaaggcaagggggctgcgagcctgcaggggagagctgagggacaccatcctagactgggaggactccctgcccgaccgggacctggcactcgccgatgaggccagcaggaacgccgacctgtccatcacgctgggtacatcgctgcagatccggcccagcgggaacctgccgctggctaccaagcgccggggaggccgcctggtcatcgtcaacctgcagcccaccaagcacgaccgccatgctgacctccgcatccatggctacgttgacgaggtcatgacccggctcatgaagcacctggggctggagatccccgcctgggacggcccccgtgtgctggagagggcgctgccacccctgccccgcccgcccacccccaagctggagcccaaggaggaatctcccacccggatcaacggctctatccccgccggccccaagcaggagccctgcgcccagcacaacggctcagagcccgccagccccaaacgggagcggcccaccagccctgccccccacagaccccccaaaagggtgaaggccaaggcggtccccagc

SEQ ID NO: 21 - DNA, 1203 bp, Artificial Sequence
  synthetic construct
  source (1)..(1203): S7
atggcagccgggggtctgagccgctccgagcgcaaagcggcggagcgggtccggaggttgcgggaggagcagcagagggagcgcctccgccaggtgtcgcgcatcctgaggaaggcggcggcggagcgcagcgccgaggagggccggctgctggccgagagcgcggacctggtaacggagctgcagggccggagccggcggcgcgagggcctgaagcggcggcaggaggaggtgtgcgacgacccggaggagctgcgggggaaggtccgggagctggccagcgccgtccggaacgccaaatacttggtcgtctacacaggcgcgggaatcagcacggcagcgtctatcccagactaccggggccctaatggagtgtggacactgcttcagaaagggagaagcgttagtgctgccgacctgagcgaggccgagccaaccctcacccacatgagcatcacccgtctgcatgagcagaagctggtgcagcatgtggtgtctcagaactgtgacgggctccacctgaggagtgggctgccgcgcacggccatctccgagctccacgggaacatgtacattgaagtctgtacctcctgcgttcccaacagggagtacgtgcgggtgttcgatgtgacggagcgcactgccctccacagacaccagacaggccggacctgccacaagtgtgggacccagctgcgggacaccattgtgcactttggggagagggggacgttggggcagcctttgaactgggaagcggcgaccgaggctgccagcagagcagacaccatcctgtgtctagggtccagcctgaaggttctaaagaagtacccacgcctctggtgcatgaccaagccccctagccggcggccgaagctttacatcgtgaacctgcagtggaccccgaaggatgactgggctgccctgaagctacatgggaagtgtgatgacgtcatgcggctcctcatggccgagctgggcttggagatccccgcctatagcaggtggcaggatcccattttctcactggcgactcccctgcgtgctggtgaagaaggcagccacagtcggaagtcgctgtgcagaagcagagaggaggccccgcctggggaccggggtgcaccgcttagctcggcccccatcctagggggctggtttggcaggggctgcacaaaacgcacaaaaaggaagaaagtgacgtaa

SEQ ID NO: 22 - DNA, 1002 bp, Artificial Sequence
  synthetic construct
  source (1)..(1002): S1A
ggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacga

SEQ ID NO: 23 - DNA, 1101 bp, Artificial Sequence
  synthetic construct
  source (1)..(1101): S1B
ggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaagggcggcggcggcagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaggcggaggatcaggcggaggctcccagtatctgtttttgccaccaaatcgttacattttccatggcgctgaggtatattcagactctgaagatgacgtc

SEQ ID NO: 24 - DNA, 1002 bp, Artificial Sequence
  synthetic construct
  source (1)..(1002): S1B1
ggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaagggcggcggcggcagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacga

SEQ ID NO: 25 - DNA, 1101 bp, Artificial Sequence
  synthetic construct
  source (1)..(1101): S1C
ggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagccgacgcggcggcggcggcggggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaggcggaggatcaggcggaggctcccagtatctgtttttgccaccaaatcgttacattttccatggcgctgaggtatattcagactctgaagatgacgtc

SEQ ID NO: 26 - DNA, 1002 bp, Artificial Sequence
  synthetic construct
  source (1)..(1002): S1C1
ggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagccgacgcggcggcggcggcggggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacga

SEQ ID NO: 27 - DNA, 1266 bp, Artificial Sequence
  synthetic construct
  source (1)..(1266): S1D
atggcggacgaggcggccctcgcccttcagcccggcggctccccctcggcggcgggggccgacagggaggccgcgtcgtcccccgccggggagccgctccgcaagaggccgcggagagatggcggcggaggatcaggaggcggcggaagcggaggcggaggctccggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaggcggaggatcaggcggaggctcccagtatctgtttttgccaccaaatcgttacattttccatggcgctgaggtatattcagactctgaagatgacgtc

SEQ ID NO: 28 - DNA, 1167 bp, Artificial Sequence
  synthetic construct
  source (1)..(1167): S1D1
atggcggacgaggcggccctcgcccttcagcccggcggctccccctcggcggcgggggccgacagggaggccgcgtcgtcccccgccggggagccgctccgcaagaggccgcggagagatggcggcggaggatcaggaggcggcggaagcggaggcggaggctccggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagttgacctcctcattgttattgggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacga

SEQ ID NO: 29 - DNA, 1266 bp, Artificial Sequence
  synthetic construct
  source (1)..(1266): S1E
atggcggacgaggcggccctcgcccttcagcccggcggctccccctcggcggcgggggccgacagggaggccgcgtcgtcccccgccggggagccgctccgcaagaggccgcggagagatggcggcggaggatcaggaggcggcggaagcggaggcggaggctccggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagccgacgcggcggcggcggcggggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacgaggcggaggatcaggcggaggctcccagtatctgtttttgccaccaaatcgttacattttccatggcgctgaggtatattcagactctgaagatgacgtc

SEQ ID NO: 30 - DNA, 1167 bp, Artificial Sequence
  synthetic construct
  source (1)..(1167): S1E1
atggcggacgaggcggccctcgcccttcagcccggcggctccccctcggcggcgggggccgacagggaggccgcgtcgtcccccgccggggagccgctccgcaagaggccgcggagagatggcggcggaggatcaggaggcggcggaagcggaggcggaggctccggtccatatacttttgttcagcaacatcttatgattggcacagatcctcgaacaattcttaaagatttattgccggaaacaatacctccacctgagttggatgatatgacactgtggcagattgttattaatatcctttcagaaccaccaaaaaggaaaaaaagaaaagatattaatacaattgaagatgctgtgaaattactgcaagagtgcaaaaaaattatagttctaactggagctggggtgtctgtttcatgtggaatacctgacttcaggtcaagggatggtatttatgctcgccttgctgtagacttcccagatcttccagatcctcaagcgatgtttgatattgaatatttcagaaaagatccaagaccattcttcaagtttgcaaaggaaatatatcctggacaattccagccatctctctgtcacaaattcatagccttgtcagataaggaaggaaaactacttcgcaactatacccagaacatagacacgctggaacaggttgcgggaatccaaaggataattcagtgtcatggttcctttgcaacagcatcttgcctgatttgtaaatacaaagttgactgtgaagctgtacgaggagatatttttaatcaggtagttcctcgatgtcctaggtgcccagctgatgaaccgcttgctatcatgaaaccagagattgtgttttttggtgaaaatttaccagaacagtttcatagagccatgaagtatgacaaagatgaagccgacgcggcggcggcggcggggtcttccctcaaagtaagaccagtagcactaattccaagttccataccccatgaagtgcctcagatattaattaatagagaacctttgcctcatctgcattttgatgtagagcttcttggagactgtgatgtcataattaatgaattgtgtcataggttaggtggtgaatatgccaaactttgctgtaaccctgtaaagctttcagaaattactgaaaaacctccacga

SEQ ID NO: 31 - DNA, 720 bp, Artificial Sequence
  synthetic construct
  source (1)..(720): C8
atggaggagagtgtcgtacggccctcagtgtttgtggtggatggacagaccgacatcccattcacgaggctgggacgaagccaccggagacagtcgtgcagtgtggcccgggtgggtctgggtctcttgctgttgctgatgggggccgggctggccgtccaaggctggttcctcctgcagctgcactggcgtctaggagagatggtcacccgcctgcctgacggacctgcaggctcctgggagcagctgatacaagagcgaaggtctcacgaggtcaacccagcagcgcatctcacaggggccaactccagcttgaccggcagcggggggccgctgttatgggagactcagctgggcctggccttcctgaggggcctcagctaccacgatggggcccttgtggtcaccaaagctggctactactacatctactccaaggtgcagctgggcggtgtgggctgcccgctgggcctggccagcaccatcacccacggcctctacaagcgcacaccccgctaccccgaggagctggagctgttggtcagccagcagtcaccctgcggacgggccaccagcagctcccgggtctggtgggacagcagcttcctgggtggtgtggtacacctggaggctggggagaaggtggtcgtccgtgtgctggatgaacgcctggttcgactgcgtgatggtacccggtcttacttcggggctttcatggtg

SEQ ID NO: 32 - DNA, 708 bp, Artificial Sequence
  synthetic construct
  source (1)..(708): C8A
atggaggagagtgtcgtacggccctcagtgtttgtggtggatggacagaccgacatcccattcacgaggctgggacgaagccaccggagacagtcgtgcagtgtggcccgggtgggtctgggtctcttgctgttgctgatgggggccgggctggccgtccaaggctggttcctcctgcagctgcactggcgtctaggagagatggtcacccgcctgcctgacggacctgcaggctcctggcaagagcgaaggtctcacgaggtcaacccagcagcgcatctcacaggggccaactccagcttgaccggcagcggggggccgctgttatgggagactcagctgggcctggccttcctgaggggcctcagctaccacgatggggcccttgtggtcaccaaagctggctactactacatctactccaaggtgcagctgggcggtgtgggctgcccgctgggcctggccagcaccatcacccacggcctctacaagcgcacaccccgctaccccgaggagctggagctgttggtcagccagcagtcaccctgcggacgggccaccagcagctcccgggtctggtgggacagcagcttcctgggtggtgtggtacacctggaggctggggagaaggtggtcgtccgtgtgctggatgaacgcctggttcgactgcgtgatggtacccggtcttacttcggggctttcatggtg

SEQ ID NO: 33 - DNA, 468 bp, Artificial Sequence
  synthetic construct
  source (1)..(468): C8B
caagagcgaaggtctcacgaggtcaacccagcagcgcatctcacaggggccaactccagcttgaccggcagcggggggccgctgttatgggagactcagctgggcctggccttcctgaggggcctcagctaccacgatggggcccttgtggtcaccaaagctggctactactacatctactccaaggtgcagctgggcggtgtgggctgcccgctgggcctggccagcaccatcacccacggcctctacaagcgcacaccccgctaccccgaggagctggagctgttggtcagccagcagtcaccctgcggacgggccaccagcagctcccgggtctggtgggacagcagcttcctgggtggtgtggtacacctggaggctggggagaaggtggtcgtccgtgtgctggatgaacgcctggttcgactgcgtgatggtacccggtcttacttcggggctttcatggtg

SEQ ID NO: 34 - protein, 242 aa, Artificial Sequence
  synthetic construct
  source (1)..(242): GPC3 scFv
DVVMTQSPLSLPVTPGEPASISCRSSQSLVHSNGNTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSS

SEQ ID NO: 35 - protein, 112 aa, Artificial Sequence
  synthetic construct
  source (1)..(112): GPC3 scFv
DVVMTQSPLSLPVTPGEPASISCRSSQSLVHSNGNTYLHWYLQKPGQSPQLLIYKVSNRFSGVPDRFSGSGSGTDFTLKISRVEAEDVGVYYCSQNTHVPPTFGQGTKLEIK

SEQ ID NO: 36 - protein, 16 aa, Artificial Sequence
  synthetic construct
  source (1)..(16): GPC3 scFvCDR1
RSSQSLVHSNGNTYLH

SEQ ID NO: 37 - protein, 7 aa, Artificial Sequence
  synthetic construct
  source (1)..(7): GPC3 scFvCDR2
KVSNRFS

SEQ ID NO: 38 - protein, 9 aa, Artificial Sequence
  synthetic construct
  source (1)..(9): GPC3 scFvCDR3
SQNTHVPPT

SEQ ID NO: 39 - protein, 115 aa, Artificial Sequence
  synthetic construct
  source (1)..(115): GPC3 scFv
QVQLVQSGAEVKKPGASVKVSCKASGYTFTDYEMHWVRQAPGQGLEWMGALDPKTGDTAYSQKFKGRVTLTADESTSTAYMELSSLRSEDTAVYYCTRFYSYTYWGQGTLVTVSS

SEQ ID NO: 40 - protein, 5 aa, Artificial Sequence
  synthetic construct
  source (1)..(5): GPC3 scFvCDR1
DYEMH

SEQ ID NO: 41 - protein, 17 aa, Artificial Sequence
  synthetic construct
  source (1)..(17): GPC3 scFvCDR2
ALDPKTGDTAYSQKFKG

SEQ ID NO: 42 - protein, 7 aa, Artificial Sequence
  synthetic construct
  source (1)..(7): GPC3 scFvCDR3
FYSYTYW

SEQ ID NO: 43 - protein, 242 aa, Artificial Sequence
  synthetic construct
  source (1)..(242): CD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 44 - protein, 107 aa, Artificial Sequence
  synthetic construct
  source (1)..(107): CD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 45 - protein, 11 aa, Artificial Sequence
  synthetic construct
  source (1)..(11): CD19 scFvCDR1
RASQDISKYLN

SEQ ID NO: 46 - protein, 7 aa, Artificial Sequence
  synthetic construct
  source (1)..(7): CD19 scFvCDR2
HTSRLHS

SEQ ID NO: 47 - protein, 9 aa, Artificial Sequence
  synthetic construct
  source (1)..(9): CD19 scFvCDR3
QQGNTLPYT

SEQ ID NO: 48 - protein, 120 aa, Artificial Sequence
  synthetic construct
  source (1)..(120): CD19 scFv
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 49 - protein, 7 aa, Artificial Sequence
  synthetic construct
  source (1)..(7): CD19 scFvCDR1
LPDYGVS

SEQ ID NO: 50 - protein, 16 aa, Artificial Sequence
  synthetic construct
  source (1)..(16): CD19 scFvCDR2
VIWGSETTYYNSALKS

SEQ ID NO: 51 - protein, 13 aa, Artificial Sequence
  synthetic construct
  source (1)..(13): CD19 scFvCDR3
HYYYGGSYAMDYW

SEQ ID NO: 52 - protein, 246 aa, Artificial Sequence
  synthetic construct
  source (1)..(246): BCMA scFv
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSS

SEQ ID NO: 53 - protein, 111 aa, Artificial Sequence
  synthetic construct
  source (1)..(111): BCMA scFv
DIVLTQSPASLAVSLGERATINCRASESVSVIGAHLIHWYQQKPGQPPKLLIYLASNLETGVPARFSGSGSGTDFTLTISSLQAEDAAIYYCLQSRIFPRTFGQGTKLEIK

SEQ ID NO: 54 - protein, 15 aa, Artificial Sequence
  synthetic construct
  source (1)..(15): BCMA scFvCDR1
RASESVSVIGAHLIH

SEQ ID NO: 55 - protein, 7 aa, Artificial Sequence
  synthetic construct
  source (1)..(7): BCMA scFvCDR2
LASNLET

SEQ ID NO: 56 - protein, 9 aa, Artificial Sequence
  synthetic construct
  source (1)..(9): BCMA scFvCDR3
LQSRIFPRT

SEQ ID NO: 57 - protein, 117 aa, Artificial Sequence
  synthetic construct
  source (1)..(117): BCMA scFv
QVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSS

SEQ ID NO: 58 - protein, 5 aa, Artificial Sequence
  synthetic construct
  source (1)..(5): BCMA scFvCDR1
DYSIN

SEQ ID NO: 59 - protein, 17 aa, Artificial Sequence
  synthetic construct
  source (1)..(17): BCMA scFvCDR2
WINTETREPAYAYDFRG

SEQ ID NO: 60 - protein, 9 aa, Artificial Sequence
  synthetic construct
  source (1)..(9): BCMA scFvCDR3
DYSYAMDYW

SEQ ID NO: 61 - protein, 259 aa, Artificial Sequence
  synthetic construct
  source (1)..(259): MSLN scFv
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSSGILGSGGGGSGGGGSGGGGSQPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLS

SEQ ID NO: 62 - protein, 116 aa, Artificial Sequence
  synthetic construct
  source (1)..(116): MSLN scFv
QPVLTQSSSLSASPGASASLTCTLRSGINVGPYRIYWYQQKPGSPPQYLLNYKSDSDKQQGSGVPSRFSGSKDASANAGVLLISGLRSEDEADYYCMIWHSSAAVFGGGTQLTVLS

SEQ ID NO: 63 - protein, 14 aa, Artificial Sequence
  synthetic construct
  source (1)..(14): MSLN scFvCDR1
TLRSGINVGPYRIY

SEQ ID NO: 64 - protein, 11 aa, Artificial Sequence
  synthetic construct
  source (1)..(11): MSLN scFvCDR2
YKSDSDKQQGS

SEQ ID NO: 65 - protein, 9 aa, Artificial Sequence
  synthetic construct
  source (1)..(9): MSLN scFvCDR3
MIWHSSAAV

SEQ ID NO: 66 - protein, 123 aa, Artificial Sequence
  synthetic construct
  source (1)..(123): MSLN scFv
QVQLQQSGPGLVTPSQTLSLTCAISGDSVSSNSATWNWIRQSPSRGLEWLGRTYYRSKWYNDYAVSVKSRMSINPDTSKNQFSLQLNSVTPEDTAVYYCARGMMTYYYGMDVWGQGTTVTVSS

SEQ ID NO: 67 - protein, 7 aa, Artificial Sequence
  synthetic construct
  source (1)..(7): MSLN scFvCDR1
SNSATWN

SEQ ID NO: 68 - protein, 18 aa, Artificial Sequence
  synthetic construct
  source (1)..(18): MSLN scFvCDR2
RTYYRSKWYNDYAVSVKS

SEQ ID NO: 69 - protein, 12 aa, Artificial Sequence
  synthetic construct
  source (1)..(12): MSLN scFvCDR3
GMMTYYYGMDVW

SEQ ID NO: 70 - protein, 241 aa, Artificial Sequence
  synthetic construct
  source (1)..(241): HER2 scFv
QVQLQQSGPELKKPGETVKISCKASGYPFTNYGMNWVKQAPGQGLKWMGWINTSTGESTFADDFKGRFDFSLETSANTAYLQINNLKSEDSATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIK

SEQ ID NO: 71 - protein, 107 aa, Artificial Sequence
  synthetic construct
  source (1)..(107): HER2 scFv
DIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIK

SEQ ID NO: 72 - protein, 11 aa, Artificial Sequence
  synthetic construct
  source (1)..(11): HER2 scFvCDR1
KASQDVYNAVA

SEQ ID NO: 73 - protein, 7 aa, Artificial Sequence
  synthetic construct
  source (1)..(7): HER2 scFvCDR2
SASSRYT

SEQ ID NO: 74 - protein, 9 aa, Artificial Sequence
  synthetic construct
  source (1)..(9): HER2 scFvCDR3
QQHFRTPFT

SEQ ID NO: 75 - protein, 119 aa, Artificial Sequence
  synthetic construct
  source (1)..(119): HER2 scFv
QVQLQQSGPELKKPGETVKISCKASGYPFTNYGMNWVKQAPGQGLKWMGWINTSTGESTFADDFKGRFDFSLETSANTAYLQINNLKSEDSATYFCARWEVYHGYVPYWGQGTTVTVSS

SEQ ID NO: 76 - protein, 5 aa, Artificial Sequence
  synthetic construct
  source (1)..(5): HER2 scFvCDR1
NYGMN

SEQ ID NO: 77 - protein, 17 aa, Artificial Sequence
  synthetic construct
  source (1)..(17): HER2 scFvCDR2
WINTSTGESTFADDFKG

SEQ ID NO: 78 - protein, 11 aa, Artificial Sequence
  synthetic construct
  source (1)..(11): HER2 scFvCDR3
WEVYHGYVPYW

SEQ ID NO: 79 - protein, 46 aa, Artificial Sequence
  synthetic construct
  source (1)..(46): CD8
TTTPAPRPPTPAPTIASQPLSLRPEACRPAAGGAVHTRGLDFACDI

SEQ ID NO: 80 - protein, 23 aa, Artificial Sequence
  synthetic construct
  source (1)..(23): CD8
YIWAPLAGTCGVLLLSLVITLYC

SEQ ID NO: 81 - protein, 41 aa, Artificial Sequence
  synthetic construct
  source (1)..(41): CD28
RSKRSRLLHSDYMNMTPRRPGPTRKHYQPYAPPRDFAAYRS

SEQ ID NO: 82 - protein, 42 aa, Artificial Sequence
  synthetic construct
  source (1)..(42): 4-1BB
KRGRKKLLYIFKQPFMRPVQTTQEEDGCSCRFPEEEEGGCEL

SEQ ID NO: 83 - protein, 112 aa, Artificial Sequence
  synthetic construct
  source (1)..(112): CD3
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - protein, 18 aa, Artificial Sequence
  synthetic construct
  source (1)..(18): T2A
EGRGSLLTCGDVEENPGP

SEQ ID NO: 85 - protein, 19 aa, Artificial Sequence
  synthetic construct
  source (1)..(19): P2A
ATNFSLLKQAGDVEENPGP

SEQ ID NO: 86 - protein, 367 aa, Artificial Sequence
  synthetic construct
  source (1)..(367): S1
GPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRGGGSGGGSQYLFLPPNRYIFHGAEVYSDSEDDV

SEQ ID NO: 87 - protein, 389 aa, Artificial Sequence
  synthetic construct
  source (1)..(389): S2
MAEPDPSHPLETQAGKVQEAQDSDSDSEGGAAGGEADMDFLRNLFSQTLSLGSQKERLLDELTLEGVARYMQSERCRRVICLVGAGISTSAGIPDFRSPSTGLYDNLEKYHLPYPEAIFEISYFKKHPEPFFALAKELYPGQFKPTICHYFMRLLKDKGLLLRCYTQNIDTLERIAGLEQEDLVEAHGTFYTSHCVSASCRHEYPLSWMKEKIFSEVTPKCEDCQSLVKPDIVFFGESLPARFFSCMQSDFLKVDLLLVMGTSLQVQPFASLISKAPLSTPRLLINKEKAGQSDPFLGMIMGLGGGMDFDSKKAYRDVAWLGECDQGCLALAELLGWKKELEDLVRREHASIDAQSGAGVPNPSTSASPKKSPPPAKDEARTTEREKPQ

SEQ ID NO: 88 - protein, 257 aa, Artificial Sequence
  synthetic construct
  source (1)..(257): S3
MVGAGISTPSGIPDFRSPGSGLYSNLQQYDLPYPEAIFELPFFFHNPKPFFTLAKELYPGNYKPNVTHYFLRLLHDKGLLLRLYTQNIDGLERVSGIPASKLVEAHGTFASATCTVCQRPFPGEDIRADVMADRVPRCPVCTGVVKPDIVFFGEPLPQRFLLHVVDFPMADLLLILGTSLEVEPFASLTEAVRSSVPRLLINRDLVGPLAWHPRSRDVAQLGDVVHGVESLVELLGWTEEMRDLVQRETGKLDGPDK

SEQ ID NO: 89 - protein, 314 aa, Artificial Sequence
  synthetic construct
  source (1)..(314): S4
MKMSFALTFRSAKGRWIANPSQPCSKASIGLFVPASPPLDPEKVKELQRFITLSKRLLVMTGAGISTESGIPDYRSEKVGLYARTDRRPIQHGDFVRSAPIRQRYWARNFVGWPQFSSHQPNPAHWALSTWEKLGKLYWLVTQNVDALHTKAGSRRLTELHGCMDRVLCLDCGEQTPRGVLQERFQVLNPTWSAEAHGLAPDGDVFLSEEQVRSFQVPTCVQCGGHLKPDVVFFGDTVNPDKVDFVHKRVKEADSLLVVGSSLQVYSGYRFILTAWEKKLPIAILNIGPTRSDDLACLKLNSRCGELLPLIDPC

SEQ ID NO: 90 - protein, 310 aa, Artificial Sequence
  synthetic construct
  source (1)..(310): S5
MRPLQIVPSRLISQLYCGLKPPASTRNQICLKMARPSSSMADFRKFFAKAKHIVIISGAGVSAESGVPTFRGAGGYWRKWQAQDLATPLAFAHNPSRVWEFYHYRREVMGSKEPNAGHRAIAECETRLGKQGRRVVVITQNIDELHRKAGTKNLLEIHGSLFKTRCTSCGVVAENYKSPICPALSGKGAPEPGTQDASIPVEKLPRCEEAGCGGLLRPHVVWFGENLDPAILEEVDRELAHCDLCLVVGTSSVVYPAAMFAPQVAARGVPVAEFNTETTPATNRFRFHFQGPCGTTLPEALACHENETVS

SEQ ID NO: 91 - protein, 355 aa, Artificial Sequence
  synthetic construct
  source (1)..(355): S6
MSVNYAAGLSPYADKGKCGLPEIFDPPEELERKVWELARLVWQSSSVVFHTGAGISTASGIPDFRGPHGVWTMEERGLAPKFDTTFESARPTQTHMALVQLERVGLLRFLVSQNVDGLHVRSGFPRDKLAELHGNMFVEECAKCKTQYVRDTVVGTMGLKATGRLCTVAKARGLRACRGELRDTILDWEDSLPDRDLALADEASRNADLSITLGTSLQIRPSGNLPLATKRRGGRLVIVNLQPTKHDRHADLRIHGYVDEVMTRLMKHLGLEIPAWDGPRVLERALPPLPRPPTPKLEPKEESPTRINGSIPAGPKQEPCAQHNGSEPASPKRERPTSPAPHRPPKRVKAKAVPS

SEQ ID NO: 92 - protein, 400 aa, Artificial Sequence
  synthetic construct
  source (1)..(400): S7
MAAGGLSRSERKAAERVRRLREEQQRERLRQVSRILRKAAAERSAEEGRLLAESADLVTELQGRSRRREGLKRRQEEVCDDPEELRGKVRELASAVRNAKYLVVYTGAGISTAASIPDYRGPNGVWTLLQKGRSVSAADLSEAEPTLTHMSITRLHEQKLVQHVVSQNCDGLHLRSGLPRTAISELHGNMYIEVCTSCVPNREYVRVFDVTERTALHRHQTGRTCHKCGTQLRDTIVHFGERGTLGQPLNWEAATEAASRADTILCLGSSLKVLKKYPRLWCMTKPPSRRPKLYIVNLQWTPKDDWAALKLHGKCDDVMRLLMAELGLEIPAYSRWQDPIFSLATPLRAGEEGSHSRKSLCRSREEAPPGDRGAPLSSAPILGGWFGRGCTKRTKRKKVT

SEQ ID NO: 93 - protein, 334 aa, Artificial Sequence
  synthetic construct
  source (1)..(334): S1A
GPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPR

SEQ ID NO: 94 - protein, 367 aa, Artificial Sequence
  synthetic construct
  source (1)..(367): S1B
GPYTFVQQHLMIGTDPRTILKDLLPETIPPPELDDMTLWQIVINILSEPPKRAAAADINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRGGGSGGGSQYLFLPPNRYIFHGAEVYSDSEDDV

SEQ ID NO: 95 - protein, 334 aa, Artificial Sequence
  synthetic construct
  source (1)..(334): S1B1
GPYTFVQQHLMIGTDPRTILKDLLPETIPPPELDDMTLWQIVINILSEPPKRAAAADINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPR

SEQ ID NO: 96 - protein, 367 aa, Artificial Sequence
  synthetic construct
  source (1)..(367): S1C
GPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEADAAAAAGSSLKVRPVALIPSSIPHEVPQILINREPLPHLHFDVELLGDCDVIINELCHRLGGEYAKLCCNPVKLSEITEKPPRGGGSGGGSQYLFLPPNRYIFHGAEVYSDSEDDV

SEQ ID NO: 97 - protein, 334 aa, Artificial Sequence
  synthetic construct
  source (1)..(334): S1C1
GPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEADAAAAAGSSLKVRPVALIPSSIPHEVPQILINREPLPHLHFDVELLGDCDVIINELCHRLGGEYAKLCCNPVKLSEITEKPPR

SEQ ID NO: 98 - protein, 422 aa, Artificial Sequence
  synthetic construct
  source (1)..(422): S1D
MADEAALALQPGGSPSAAGADREAASSPAGEPLRKRPRRDGGGGSGGGGSGGGGSGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPRGGGSGGGSQYLFLPPNRYIFHGAEVYSDSEDDV

SEQ ID NO: 99 - protein, 389 aa, Artificial Sequence
  synthetic construct
  source (1)..(389): S1D1
MADEAALALQPGGSPSAAGADREAASSPAGEPLRKRPRRDGGGGSGGGGSGGGGSGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEVDLLIVIGSSLKVRPVALIPSSIPHEVPQILINREPLPHLHFDVELLGDCDVIINELCHRLGGEYAKLCCNPVKLSEITEKPPR

SEQ ID NO: 100 - protein, 422 aa, Artificial Sequence
  synthetic construct
  source (1)..(422): S1E
MADEAALALQPGGSPSAAGADREAASSPAGEPLRKRPRRDGGGGSGGGGSGGGGSGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEADAAAAAGSSLKVRPVALIPSSIPHEVPQILINREPLPHLHFDVELLGDCDVIINELCHRLGGEYAKLCCNPVKLSEITEKPPRGGGSGGGSQYLFLPPNRYIFHGAEVYSDSEDDV

SEQ ID NO: 101 - protein, 389 aa, Artificial Sequence
  synthetic construct
  source (1)..(389): S1E1
MADEAALALQPGGSPSAAGADREAASSPAGEPLRKRPRRDGGGGSGGGGSGGGGSGPYTFVQQHLMIGTDPRTILKDLLPETIPPPELDDMTLWQIVINILSEPPKRKKRKDINTIEDAVKLLQECKKIIVLTGAGVSVSCGIPDFRSRDGIYARLAVDFPDLPDPQAMFDIEYFRKDPRPFFKFAKEIYPGQFQPSLCHKFIALSDKEGKLLRNYTQNIDTLEQVAGIQRIIQCHGSFATASCLICKYKVDCEAVRGDIFNQVVPRCPRCPADEPLAIMKPEIVFFGENLPEQFHRAMKYDKDEADAAAAAGSSLKVRPVALIPSSIPHEVPQILINREPLPHLHFDVELLGDCDVIINELCHRLGGEYAKLCCNPVKLSEITEKPPR

SEQ ID NO: 102 - protein, 240 aa, Artificial Sequence
  synthetic construct
  source (1)..(240): C8
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 103 - protein, 236 aa, Artificial Sequence
  synthetic construct
  source (1)..(236): C8A
MEESVVRPSVFVVDGQTDIPFTRLGRSHRRQSCSVARVGLGLLLLLMGAGLAVQGWFLLQLHWRLGEMVTRLPDGPAGSWQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 104 - protein, 156 aa, Artificial Sequence
  synthetic construct
  source (1)..(156): C8B
QERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 105 - protein, 8 aa, Artificial Sequence
  synthetic construct
  source (1)..(8): SIRT1
PLRKRPRR

SEQ ID NO: 106 - protein, 8 aa, Artificial Sequence
  synthetic construct
  source (1)..(8): SIRT1
PPKRKKRK

SEQ ID NO: 107 - protein, 8 aa, Artificial Sequence
  synthetic construct
  source (1)..(8): SIRT1
PLRKRPAA

SEQ ID NO: 108 - protein, 8 aa, Artificial Sequence
  synthetic construct
  source (1)..(8): SIRT1
PPKRAAAA

SEQ ID NO: 109 - protein, 8 aa, Artificial Sequence
  synthetic construct
  source (1)..(8): SIRT1
LLLTDGLL

SEQ ID NO: 110 - protein, 7 aa, Artificial Sequence
  synthetic construct
  source (1)..(7): SIRT1
VDLLIVI

SEQ ID NO: 111 - protein, 8 aa, Artificial Sequence
  synthetic construct
  source (1)..(8): SIRT1
AAATDGAA

SEQ ID NO: 112 - protein, 7 aa, Artificial Sequence
  synthetic construct
  source (1)..(7): SIRT1
ADAAAAA

(19) United States
(12) Patent Application Publication
(10) Pub. No.: US 2025/0114400 A1
(43) Pub. Date: Apr. 10, 2025

(54) Application of SIRT1-7 protein in immunotherapy

(71) Applicant: Avenex Biosciences LTD., Hongkong (CN)

(72) Inventors: Youguo LING, Nanjing (CN); Haotian WU, Nanjing (CN)

(73) Assignee: Avenex Biosciences LTD., Hongkong (CN)

(21) Appl. No.: 18/725,839

(22) PCT Filed: Dec. 30, 2021
(86) PCT No.: PCT/CN2021/143151

§ 371 Date: Jul. 1, 2024

(51) Int. Cl. A61K 35/17; A61K 40/31; A61K 40/32; A61K 40/42; A61P 35/00; C07K 14/705; C07K 16/28; C07K 16/30; C07K 16/32; C12N 5/00; C12N 5/0783; C12N 9/10; C12N 15/86

(52) U.S. Cl. CPC A61K 35/17; A61K 40/31; A61K 40/32; A61K 40/4205; A61K 40/4211; A61K 40/4215; A61K 40/4224; A61K 40/4244; A61K 40/4255; A61K 40/4261; A61P 35/00; C07K 14/70575; C07K 16/2803; C07K 16/2878; C07K 16/303; C07K 16/32; C12N 5/0018; C12N 5/0636; C12N 9/1029; C12N 15/86; A61K 2239/13; A61K 2239/17; A61K 2239/21; A61K 2239/22; C12N 2740/15043; C12Y 203/01

(57) ABSTRACT

An application of a combination of SIRT1-7 protein or CD258 protein and SIRT1-7 protein for promoting immune cell proliferation is provided.

### CROSS REFERENCE TO THE RELATED APPLICATIONS

This application is the national phase entry of International Application No. PCT/CN2021/143151, filed on Dec. 30, 2021, the entire contents of which are incorporated herein by reference.

### SEQUENCE LISTING

The present application contains a Sequence Listing which has been submitted in ASCII format via EFS-Web and is hereby incorporated by reference in its entirety. Said ASCII copy is named LZ0101P_Sequence_Listing_20240628.txt, created Jun. 28, 2024, and is 129,340 bytes in size.

### TECHNICAL FIELD

The present invention relates to the field of immunology. Specifically, the present invention relates to the application of one or more SIRT1-7 proteins or their functional mutants, or the combination of CD258 proteins or their functional mutants and one or more SIRT1-7 proteins or their functional mutants in immunotherapy.

### BACKGROUND

Chimeric antigen receptor (CAR) T cell therapy is a new generation of tumor immunotherapy technology that has developed rapidly in recent years. The structure of CAR is mainly composed of four parts: a single chain variable fragment (scFv) that specifically recognizes tumor-associated antigens (TAA), a hinge region, a transmembrane region, and an intracellular signal transduction region. The scFv is generally an antigen binding region composed of the heavy chain variable region (VH) and the light chain variable region (VL) of a monoclonal antibody which are connected by a polypeptide linker, namely VH-Linker-VL or VL-Linker-VH; the hinge region is usually selected from the immunoglobulin superfamily, such as IgG4, CD8, IgG1 etc.; the transmembrane region generally comprises CD8, CD28 or CD4, etc.; the intracellular signal transduction region mainly comprises CD3ζ chain and co-stimulatory signal molecules such as 41BB, CD28, ICOS, OX40, etc. Therefore, T cells that express CAR can directly recognize TAA without going through the antigen presentation mechanism, and have the unique advantage of MHC (Major histocompatibility complex) independence.

CAR-T technology has performed well in the treatment of hematological tumors. With CD19 as the target, CAR-T has achieved amazing results in the treatment of acute and chronic B-lymphocytic leukemia. However, the clinical efficacy of CAR-T on solid tumors is very limited, and new solutions are urgently needed.

Sirtuins are a class of proteins whose activities of deacetylase and ADP-ribosyltransferase depend on NAD⁺ and whose core regions are highly conserved. They play an important role in regulating the gene expression of histone acetylation/deacetylation. There are mainly seven Sirtuin protein subtypes in mammals, namely SIRT1 protein, SIRT2 protein, SIRT3 protein, SIRT4 protein, SIRT5 protein, SIRT6 protein, SIRT7 protein, sometimes “SIRT1-7” represents the 7 type SIRT proteins. SIRT1 protein, SIRT6 protein and SIRT7 protein are located in the cell nucleus and mainly regulate biological processes such as transcription, the structure of terminal centromere of chromatin, aging and metabolism etc. In addition, SIRT1 protein also exists in cytoplasm and mitochondria. SIRT2 protein mainly locates in the cytoplasm, and interacts with cytoplasmic microtubules which is one of the important factors regulating cell apoptosis; SIRT3 protein, SIRT4 protein and SIRT5 protein locate in the mitochondria, mainly regulate the acetylation modification of the key proteins in the process of cellular energy metabolism, and plays an important role in biological processes such as cellular oxidative phosphorylation, tricarboxylic acid cycle, aerobic oxidation of fatty acids and amino acid degradation.

CD258 protein is also known as tumor necrosis factor superfamily member 14 (TNFSF14), homologous to lymphotoxin, inducible expression, competes with herpes simplex virus (HSV) glycoprotein D for HSV entry mediator (HVEM), a receptor expressed on T lymphocytes, LIGHT] or HVEM ligand (Herpes virus entry mediator-ligand, HVEM-L). CD258 protein mainly expressed on the surface of activated T cells, B cells, natural killer (NK) cells, immature dendritic cells (im DC) and monocytes, and CD258 protein has three forms: full-length type 2 transmembrane glycoprotein containing 240 amino acid residues and a relative molecular mass of 29 ku; non-glycosylated protein composed of 204 amino acid residues and lacking the transmembrane region and only activating T lymphocytes; and soluble CD258 protein under the shearing action of cell surface metalloenzyme. The signaling pathway mediated by CD258 protein is closely related to the occurrence and development of inflammatory and autoimmune diseases, graft-versus-host disease, pathogen infection, and immune regulation of tumors.

### SUMMARY

The technical problem solved by the present invention is the application of one or more of SIRT1-7 protein or the functional mutant thereof, one or more of CD258 protein or the functional mutant thereof in immunotherapy, which is specifically manifested in promoting the formation of memory T cells, inhibiting the expression of immune negative regulatory proteins in immune cells, enhancing the release of cytokines in immune cells, enhancing the killing ability of immune cells against tumor cells, and mobilizing the anti-tumor immune response of human body, solving the problem of tumor heterogeneity and preventing tumor recurrence.

Specifically, on the one hand, the present application provides a method for promoting the proliferation of immune cells, which comprises upregulating the expression of one or more of SIRT1-7 proteins or functional mutants thereof, and CD258 proteins or functional mutants thereof in the immune cells.

On the one hand, the present application provides a method for promoting the generation of memory immune cells, which comprises upregulating the expression of one or more of SIRT1-7 proteins or functional mutants thereof, and CD258 proteins or functional mutants thereof in the immune cells, thereby promoting the differentiation of the immune cells into memory immune cells.

On the one hand, the present application provides a method for inhibiting the differentiation of immune cells, which comprises upregulating the expression of one or more of SIRT1-7 proteins or functional mutants thereof, and CD258 proteins or functional mutants thereof in the immune cells, thereby inhibiting the differentiation of the immune cells into differentiated immune cells.

On the one hand, the present application provides a method for inhibiting the expression of immune negative regulatory proteins in immune cells, which comprises upregulating the expression of one or more of SIRT1-7 proteins or functional mutants thereof, and CD258 proteins or functional mutants thereof in the immune cells, thereby inhibiting the expression of immune negative regulatory proteins in the immune cells. In certain embodiments, the immune negative regulatory proteins are selected from the group consisting of PD1, PDL1, TIM3 and LAG3.

On the one hand, the present application provides a method for enhancing the release of cytokines by immune cells, which comprises upregulating the expression of one or more of SIRT1-7 protein or functional mutants thereof, and CD258 proteins or functional mutants thereof in the immune cells.

In certain embodiments, the cytokine comprises interleukin, interferon and/or tumor necrosis factor. In certain embodiments, the cytokine comprises IL-2, IL4, IL6, IL7, IL10, IL12, TNF-α and/or IFNγ.

On the one hand, the present application provides a method for enhancing the tumor killing ability of immune cells, which comprises up-regulating the expression of SIRT1-7 protein or functional mutants thereof, and CD258 proteins or functional mutants thereof, and the combination of SIRT1-7 protein or functional mutant thereof and CD258 protein or functional mutant thereof.

On the one hand, the present application provides a method for solving tumor heterogeneity, the method comprising: administering immune cells to a subject, wherein the expression of one or more of SIRT1-7 protein or functional mutants thereof, and CD258 proteins or functional mutants thereof in the immune cells is up-regulated.

On the one hand, the present application provides a method for preventing tumor recurrence in a subject, the method comprising: administering immune cells to the subject, wherein the expression of one or more of SIRT1-7 protein or functional mutants thereof, and CD258 proteins or functional mutants thereof in the immune cells is upregulated.

On the one hand, the present application provides a method for treating a tumor in a subject in need, comprising the following steps: administering immune cells to the subject, wherein the expression of one or more of SIRT1-7 protein or functional mutants thereof, and CD258 proteins or functional mutants thereof in the immune cells is upregulated.

In certain embodiments, the tumor comprises liver cancer, lung cancer, leukemia and mesothelioma.

In certain embodiments, the method is in vivo or in vitro.

In one or more embodiments of the present invention, the expression of SIRT1-7 protein or functional mutants thereof, and CD258 proteins or functional mutants thereof is upregulated.

In one or more embodiments of the invention, the immune cell is a lymphocyte. In some embodiments, the immune cells are T cells, B cells, natural killer (NK) cells, immature dendritic cells (im DC), monocytes and macrophages. In some embodiments, the T cells comprise memory stem cell-like T cells (TSCM) and/or central memory T cells (TCM). In some embodiments, the TSCM is CCR7⁺ and/or CD62L⁺. In some embodiments, the TSCM also has one or more properties selected from the following group: CD45RA⁺ or CD45RA⁻, CD45RO⁺ or CD45RO⁻, CD27⁺, CD28⁺, CD127⁺, CD122⁺, CD95⁺, CD3⁺, CD4⁺ and CD8⁺.

In this application, the term “memory immune cells” generally refers to immune cells with immune memory. The immune memory may refer to the ability to produce a rapid and strong immune response when encountering the same antigen again after specific recognition and response to a certain antigen. In the present application, the immunological memory cells may include memory T cells. The memory T cells may be divided into memory stem cell-like T cells (TSCM) and central memory T cells (TCM).

In the present application, the term “differentiated immune cells” generally refers to immune cells with a certain degree of differentiation. For example, the differentiated immune cells may be T cells with a certain degree of differentiation. In the present application, the differentiated immune cells may be obtained by culturing the immune cells to differentiate them to a certain degree. For example, the differentiated immune cells may comprise regulatory T cells (Treg).

In the present application, the term “regulatory T cell” (Treg) generally refers to a group of lymphocytes that negatively regulate the body's immune response. The molecular marker of the regulatory T cell can be transcription factor Foxp3 or CD127⁻. In the present application, the regulatory T cells can be divided into two categories: naturally existing and induced regulatory T cells. Wherein, naturally existing cells are CD4⁺CD25⁺ cells, and induced cells are TR1 cells and TH3 cells.

In the present application, the term “T memory stem cells, TSCM) generally refers to cells that are in the early differentiation stage of memory T cells, which have the characteristics of stem cells and have a strong potential for multidirectional differentiation. After responding to antigen stimulation, TSCM cells can differentiate into central memory T cells (Central memory T cells, TCM), effector memory T cells (TEM) and effector T cells (TEF).

In this application, the term “central memory T cells” (TCM) generally refers to T cells with long-term memory generated by naive T cells which are activated by antigens. The biomarkers of the TCM may include CD62L⁺ and CD45RO⁺. The central memory T cells can pass through the lymphatic shield and return to the lymph nodes while being activated by antigens.

In certain embodiments, the immune cells are selected from genetically modified immune cells, and the genetically modified immune cells express chimeric antigen receptors (CARs) or T cell receptors (TCRs). In certain embodiments, the genetically modified immune cells are genetically modified T cells.

In certain embodiments, the TCR comprises the subunits that are selected from TCRα, TCRβ, TCRγ, and TCRδ.

In certain embodiments, the subunits of the TCR include extracellular domain-variable regions that specifically bind to and/or recognize tumor antigens. In certain embodiments, the extracellular domain-variable region comprises: TCRα variable region fragment Vα, TCRα variable region fragment Jα, TCRβ variable region fragment Vβ, TCRβ variable region fragment Dβ and TCRβ variable region fragment Jβ.

In certain embodiments, the extracellular domain-variable region specifically binds and/or recognizes a target that comprises: MAGEA family members, CTA family members, HPV virus and tyrosinase.

In certain embodiments, the extracellular domain variable region specifically binds and/or recognizes a target that comprises: MAGEA3, MAGEA4, NY-ESO-1, MART1, HPV16-E6 and melanoma antigen tyrosinase.

In certain embodiments, the CAR comprises an intracellular domain, which includes a signaling domain and/or a co-stimulatory domain.

In certain embodiments, the signaling domain comprises: the signaling domain of CD3ζ (preferably the nucleotide sequence thereof is shown as SEQ ID NO:11, the amino acid sequence thereof is shown as SEQ ID NO:83), the signaling domain of CD38, and the signaling domain of CD38. In certain embodiments, the signaling domain comprises the sequence as shown in SEQ ID NO:83 or a homologous sequence having at least 80%, at least 85%, at least 90%, at least 91%, at least 92%, at least 93%, at least 94%, at least 95%, at least 96%, at least 97%, at least 98% or at least 99% homology thereto consists of it, and the homologous sequence still has the function of the sequence as shown in SEQ ID NO:83.

The term “signaling domain” generally refers to the functional signaling domain of a protein that comprises CD3ζ, CD3γ, CD3δ, CD3ε, FcRγ (FCER1G), FcRβ (Fc Epsilon R1b), CD79a, CD79b, FcγRIIa, DAP10 and DAP12. In the present application, the signaling domain may comprise: CD3ζ, CD3δ and CD3ε.

In certain embodiments, the costimulatory domain comprises: the costimulatory domain of CD27, the costimulatory domain of CD28 (preferably the nucleotide sequence thereof is shown as SEQ ID NO: 9, the amino acid sequence thereof is shown as SEQ ID NO:81) and the costimulatory domain of 4-1BB (preferably the nucleotide sequence thereof is shown as SEQ ID NO:10, the amino acid sequence thereof is shown as SEQ ID NO:82). In certain embodiments, the co-stimulatory domain comprises or consists of a sequence that is shown as any of the following or a sequence having at least 80%, at least 85%, at least 90%, at least 91%, at least 92%, at least 93%, at least 94%, at least 95%, at least 96%, at least 97%, at least 98% or at least 99% homology thereto: SEQ ID NO:81 and SEQ ID NO: 82. The homologous sequences still have the function of the sequences shown in SEQ ID NO:81 or 82.

The term “costimulatory domain” generally refers to the functional signaling domain of one or more of proteins that are selected from the following: CD27, CD28, 4-1BB (CD137), OX40, CD30, CD40, PD-1, ICOS, lymphocyte function-associated antigen 1 (LFA-1), CD2, CD7, LIGHT, NKG2C, B7-H3, the ligand that specifically binds to CD83, CDS, ICAM-1, GITR, BAFFR, HVEM (LIGHTR), SLAMF7, NKp80 (KLRF1), CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CD11d, ITGAE, CD103, ITGAL, CD 11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46 and NKG2D. In the present application, the co-stimulatory domain may include: CD27, CD28 and 4-1BB.

In certain embodiments, the CAR comprises a hinge region. In certain embodiments, the hinge region comprises the hinge region of IgG4, the hinge region of IgG1 and the hinge region of CD8 (preferably the nucleotide sequence thereof is shown as SEQ ID NO:7, and the amino acid sequence thereof is shown as SEQ ID NO:79). In certain embodiments, the hinge region comprises or consists of the sequence as shown in SEQ ID NO:79.

The term “hinge region” generally refers to the region between the CH1 and CH2 functional regions of the heavy chain in immunoglobulin. The hinge region is a region located between the scFv and the cytomembrane of T cell. The hinge region is generally selected from the IgG family, for example, it can be selected from IgG1 and IgG4, and can also be selected from IgD and CD8. In the present application, the hinge region may include a portion that is selected from the group consisting of the hinge region of IgG4, the hinge region of IgG1 and the hinge region of CD8.

In certain embodiments, the CAR comprises a transmembrane region. In certain embodiments, the transmembrane region comprises the transmembrane region of CD8 (preferably the nucleotide sequence thereof is shown as SEQ ID NO: 8, and the amino acid sequence thereof is shown as SEQ ID NO: 80), the transmembrane region of CD28, and the transmembrane region of CD24. In certain embodiments, the transmembrane region comprises or consists of the sequence that is shown as SEQ ID NO: 8.

The term “transmembrane region”, which is generally composed of dimeric membrane proteins, generally refers to the transmembrane region connecting the extracellular antigen binding domain and the intracellular signaling domain, mainly comprises CD3ζ, CD4, CD8, CD28, etc., and can anchor the CAR structure to the cytomembrane of T cell. Different designs of the transmembrane region can affect the expression of the introduced CAR gene. In the present application, the transmembrane region may comprise the portion selected from the following groups: the transmembrane region of CD8, the transmembrane region of CD28, and the transmembrane region of CD24.

In certain embodiments, the CAR comprises a targeting portion. In certain embodiments, the targeting portion is scFv. Preferably, the targeting moiety comprises GPC3-targeting scFv, CD19-targeting scFv, BCMA-targeting scFv, MSLN-targeting scFv, and HER2-targeting scFv.

In some embodiments, the GPC3-targeting scFv comprises LCDR1 (preferably according to the Kabat numbering system, the sequence thereof is shown in SEQ ID NO:36), LCDR2 (preferably according to the Kabat numbering system, the sequence thereof is shown in SEQ ID NO:37) and LCDR3 (preferably according to the Kabat numbering system, the sequence thereof is shown in SEQ ID NO: 38) that are contained in the light chain variable region which is shown as SEQ ID NO:35, and HCDR1 (preferably according to the Kabat numbering system, the sequence thereof is shown in SEQ ID NO: 40), HCDR2 (preferably according to the Kabat numbering system, the sequence thereof is shown in SEQ ID NO:41) and HCDR3 (preferably according to the Kabat numbering system, the sequence thereof is shown in SEQ ID NO:42) that are contained in the heavy chain variable region which is shown as SEQ ID NO:42. More preferably, the GPC3-targeting scFv comprises the nucleotide sequence that is shown as SEQ ID NO:2 or the amino acid sequence that is shown as SEQ ID NO:34, or the homology sequence having at least 80%, at least 85%, at least 90%, at least 91%, at least 92%, at least 93%, at least 94%, at least 95%, at least 96%, at least 97%, at least 98% or at least 99% homology thereto, or is composed of the same, and the homologous sequence still has the function of the sequence shown in SEQ ID NO:2 or 34.

In some embodiments, the CD19-targeting scFv comprises LCDR1 (preferably according to the Kabat numbering system, the sequence thereof is shown in SEQ ID NO:45), LCDR2 (preferably according to the Kabat numbering system, the sequence thereof is shown in SEQ ID NO:46) and LCDR3 (preferably according to the Kabat numbering system, the sequence thereof is shown in SEQ ID NO: 47) that are contained in the light chain variable region which is shown as SEQ ID NO:44, and HCDR1 (preferably according to the Kabat numbering system, the sequence thereof is shown in SEQ ID NO: 49), HCDR2 (preferably according to the Kabat numbering system, the sequence thereof is shown in SEQ ID NO:50) and HCDR3 (preferably according to the Kabat numbering system, the sequence thereof is shown in SEQ ID NO:51) that are contained in the heavy chain variable region which is shown as SEQ ID NO:48. More preferably, the CD19-targeting scFv comprises the nucleotide sequence that is shown as SEQ ID NO:3 or the amino acid sequence that is shown as SEQ ID NO:43, or the homology sequence having at least 80%, at least 85%, at least 90%, at least 91%, at least 92%, at least 93%, at least 94%, at least 95%, at least 96%, at least 97%, at least 98% or at least 99% homology thereto, or is composed of the same, and the homologous sequence still has the function of the sequence shown in SEQ ID NO:3 or 43.

In some embodiments, the BCMA-targeting scFv comprises LCDR1 (preferably according to the Kabat numbering system, the sequence thereof is shown in SEQ ID NO:54), LCDR2 (preferably according to the Kabat numbering system, the sequence thereof is shown in SEQ ID NO:55) and LCDR3 (preferably according to the Kabat numbering system, the sequence thereof is shown in SEQ ID NO: 56) that are contained in the light chain variable region which is shown as SEQ ID NO:53, and HCDR1 (preferably according to the Kabat numbering system, the sequence thereof is shown in SEQ ID NO: 58), HCDR2 (preferably according to the Kabat numbering system, the sequence thereof is shown in SEQ ID NO:59) and HCDR3 (preferably according to the Kabat numbering system, the sequence thereof is shown in SEQ ID NO:60) that are contained in the heavy chain variable region which is shown as SEQ ID NO:57. More preferably, the BCMA-targeting scFv comprises the nucleotide sequence that is shown as SEQ ID NO:4 or the amino acid sequence that is shown as SEQ ID NO: 52, or the homology sequence having at least 80%, at least 85%, at least 90%, at least 91%, at least 92%, at least 93%, at least 94%, at least 95%, at least 96%, at least 97%, at least 98% or at least 99% homology thereto, or is composed of the same, and the homologous sequence still has the function of the sequence shown in SEQ ID NO:4 or 52.

In some embodiments, the MSLN-targeting scFv comprises LCDR1 (preferably according to the Kabat numbering system, the sequence thereof is shown in SEQ ID NO:63), LCDR2 (preferably according to the Kabat numbering system, the sequence thereof is shown in SEQ ID NO:64) and LCDR3 (preferably according to the Kabat numbering system, the sequence thereof is shown in SEQ ID NO: 65) that are contained in the light chain variable region which is shown as SEQ ID NO:62, and HCDR1 (preferably according to the Kabat numbering system, the sequence thereof is shown in SEQ ID NO: 67), HCDR2 (preferably according to the Kabat numbering system, the sequence thereof is shown in SEQ ID NO:68) and HCDR3 (preferably according to the Kabat numbering system, the sequence thereof is shown in SEQ ID NO:69) that are contained in the heavy chain variable region which is shown as SEQ ID NO:66. More preferably, the MSLN-targeting scFv comprises the nucleotide sequence that is shown as SEQ ID NO:5 or the amino acid sequence that is shown as SEQ ID NO: 61, or the homology sequence having at least 80%, at least 85%, at least 90%, at least 91%, at least 92%, at least 93%, at least 94%, at least 95%, at least 96%, at least 97%, at least 98% or at least 99% homology thereto, or is composed of the same, and the homologous sequence still has the function of the sequence shown in SEQ ID NO:5 or 61.

In some embodiments, the HER2-targeting scFv comprises LCDR1 (preferably according to the Kabat numbering system, the sequence thereof is shown in SEQ ID NO:72), LCDR2 (preferably according to the Kabat numbering system, the sequence thereof is shown in SEQ ID NO:73) and LCDR3 (preferably according to the Kabat numbering system, the sequence thereof is shown in SEQ ID NO: 74) that are contained in the light chain variable region which is shown as SEQ ID NO:71, and HCDR1 (preferably according to the Kabat numbering system, the sequence thereof is shown in SEQ ID NO: 76), HCDR2 (preferably according to the Kabat numbering system, the sequence thereof is shown in SEQ ID NO:77) and HCDR3 (preferably according to the Kabat numbering system, the sequence thereof is shown in SEQ ID NO:78) that are contained in the heavy chain variable region which is shown as SEQ ID NO:75. More preferably, the HER2-targeting scFv comprises the nucleotide sequence that is shown as SEQ ID NO:6 or the amino acid sequence that is shown as SEQ ID NO: 70, or the homology sequence having at least 80%, at least 85%, at least 90%, at least 91%, at least 92%, at least 93%, at least 94%, at least 95%, at least 96%, at least 97%, at least 98% or at least 99% homology thereto, or is composed of the same, and the homologous sequence still has the function of the sequence shown in SEQ ID NO:6 or 70.

In certain embodiments, the targeting moiety specifically binds and/or recognizes the antigen of a tumor. In certain embodiments, the targeting moiety specifically binds and/or recognizes a target that comprises the surface antigen of a B lymphocyte, a TNF family member, a HER family member, and a GPC family member. In certain embodiments, the targeting moiety specifically binds and/or recognizes a target that comprises CD19, BCMA, HER2, Mesothelin, and GPC3. In certain embodiments, the targeting portion comprises a sequence that is shown as any one of the following: SEQ ID NO: 2, 3, 4, 5, 6, 34, 43, 52, 61 or 70, or a homology sequence having at least 80%, at least 85%, at least 90%, at least 91%, at least 92%, at least 93%, at least 94%, at least 95%, at least 96%, at least 97%, at least 98% or at least 99% homology thereto, or is composed thereof, and the homologous sequence still has the function of the sequence shown in SEQ ID NO: 2, 3, 4, 5, 6, 34, 43, 52, 61 or 70.

The term “tumor antigen” generally refers to an antigenic substance in a tumor cell or produced by a tumor cell, which has the ability to trigger an immune response in a host. For example, a tumor antigen may be a protein, polypeptide, peptide, or fragment thereof that constitutes a part of a tumor cell and is capable of inducing tumor-specific cytotoxic T lymphocytes. The tumor antigen peptides may be peptides produced as a result of tumor antigen degradation in tumor cells and may induce or activate tumor-specific cytotoxic T lymphocytes after being expressed on the cell surface by binding to HLA molecules. In some embodiments, the term “tumor antigen” may also refer to biological molecules (e.g., proteins, carbohydrates, glycoproteins, etc.) that are specifically or preferentially or differentially expressed on cancer cells and/or associated with cancer cells, thereby providing a cancer-preferred or specific target. For example, the preferential expression may be a conventional preferential expression, or preferential expression in a specific region of the organism (e.g., within a specific organ or tissue), with respect to any other cell in the organism. For example, the tumor antigen may comprise TSHR, CD19, CD123, CD138, CD22, CD30, CD171, CS-1, CLL-1, CD33, EGFRvIII, GD2, GD3, BCMA, TnAg, PSMA, ROR1, FLT3, FAP, TAG72, CD38, CD44v6, CEA, EPCAM, B7H3, KIT, IL-13Ra2, mesothelin, IL-11Ra, PSCA, PRSS21, VEGFR2, LewisY, CD24, PDGFR-β, SSEA-4, CD20, folate receptor α, ERBB2 (Her2/neu), MUC1, EGFR, NCAM, Prostase, PAP, ELF2M, Ephrin B2, I GF-I receptor, CAIX, LMP2, gp100, bcr-abl, tyrosinase, EphA2, fucosyl GM1, sLe, GM3, TGS5, HMWMAA, o-acetyl-GD2, folate receptor β, TEM1/CD248, TEM7R, CLDN6, GPRC5D, CXORF61, CD97, CD179a, ALK, polysialic acid, PLACI, GloboH, NY-BR-1, UPK2, HAVCR1, ADRB3, PANX3, GPR20, LY6K, OR51E2, T ARP, WT1, NY-ESO-1, LAGE-la, MAGE-A1, legumain, HPVE6, E7, MAGE A1, ETV6-AML, sperm protein 17, XAGE1, ie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2ETS fusion Gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxylesterase, mut hsp702, CD79a, CD79b, CD72, LAIR1, FCAR, LILRA2, CD300LF, CLEC12A, BST2, EMR2, LY75, GPC3, FCRL5 and IGLL1.

In certain embodiments, the method further comprises the following steps: isolating and obtaining peripheral blood mononuclear cells PBMC, CD3 T lymphocytes, CD8 T lymphocytes, CD4 T lymphocytes or regulatory T cells.

In certain embodiments, the method further comprises: adding one or more T cell stimulating factors to the separated PBMC. In some embodiments, the T cell stimulating factor comprises: antibodies that targets the surface antigens of B lymphocytes, anti-TNF antibodies, intracellular polyesters and antibiotics. In some embodiments, the T cell stimulating factor comprises: anti-CD3 antibodies, anti-CD28 antibodies, anti-4-1BB antibodies, anti-CD80 antibodies, anti-CD86 antibodies, PHA, PMA and ionomycin.

In some embodiments, the T cell stimulating factor is an anti-CD3 antibody, and the concentration of the anti-CD3 antibody is 1-10000 ng/mL. In some embodiments, the T cell stimulating factor is an anti-CD28 antibody, and the concentration of the anti-CD28 antibody is 1-10000 ng/ml.

In some embodiments, the method further comprises: adding one or more cytokines to the separated PBMC.

In some embodiments, the cytokine is an interleukin.

The term “interleukin” generally refers to a secretory protein or signaling molecule that can promote the development and differentiation of T and/or B lymphocytes and/or hematopoietic cells. Interleukins can be synthesized by auxiliary CD4 T lymphocytes, monocytes, macrophages and endothelial cells. As used herein, interleukins (IL) can comprise IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35 and/or IL-36. As used herein, the term “interleukin” may include full-length interleukins or fragments thereof (e.g., truncated forms) or variants thereof that substantially retain the biological activity of the corresponding wild-type interleukin (e.g., having a biological activity of at least 80%, at least 85%, at least 90%, at least 91%, at least 92%, at least 93%, at least 94%, at least 95%, at least 96%, at least 97%, at least 98%, at least 99%, or even 100% of the biological activity of the corresponding wild-type interleukin). The interleukins used herein may be from any mammalian species. In certain embodiments, the interleukin is from a species comprising humans, horses, cows, mice, pigs, rabbits, cats, dogs, rats, goats, sheep, and non-human primates. In certain embodiments, the interleukin may be in a mutant form. For example, the interleukin may be super IL-2 (also known as sIL2, referring to Nature 484, 529-533, 2612), which can be obtained by modifying IL-2 to increase its binding affinity to IL-2RB. Mutations in sIL-2 are mainly in the core of cytokines, and molecular dynamics simulations show that evolutionary mutations make IL-2 stable, reducing the flexibility of the helix in the IL-2RB binding site to a conformation similar to the optimized receptor binding when bound to CD25. Compared with IL-2, sIL-2 induces excellent expansion of cytotoxic T cells, resulting in improved anti-tumor response in vivo, causing less expansion of T regulatory cells, and reduced pulmonary edema. For example, in the present application, the cytokine may comprise IL-2, IL4, IL6, IL7, IL10, IL21, TNF-α and/or IFNγ.

In certain embodiments, the interleukin comprises: IL2, IL21, IL7 and IL15. In certain embodiments, the interleukin is IL2, and the concentration of the IL2 is 0.1-10000 U/mL. In certain embodiments, the interleukin is IL21, and the concentration of the IL21 is 0.01-1000 ng/ml. In certain embodiments, the interleukin is IL7, and the concentration of IL7 is 0.01-1000 ng/ml. In certain embodiments, the interleukin is IL15, and the concentration of IL15 is 0.01-1000 ng/mL.

In certain embodiments, the SIRT1-7 or functional mutants thereof, CD258 or functional mutants thereof are from humans.

SIRT1 protein has two nuclear localization signals PLRKRPRR (shown as SEQ ID NO:105) and PPKRKKRK (shown as SEQ ID NO:106), the preferred mutation is the substitution of any amino acid with A or deletion of any amino acid, the more preferred mutants are PLRKRPAA (shown as SEQ ID NO: 107) and PPKRAAAA (shown as SEQ ID NO:108); the preferred deletion mutation is the complete deletion of PLRKRPRR (shown as SEQ ID NO:105) and PPKRKKRK (shown as SEQ ID NO: 106); SIRT1 protein has two Nuclear Export Signal: LLLTDGLL (shown as SEQ ID NO:10 9 and VDLLIVI (shown as SEQ ID NO: 110), preferably any amino acid is replaced by A or deleted, more preferably the mutation are AAATDGAA (shown as SEQ ID NO: 111) and ADAAAAA (shown as SEQ ID NO: 112) (see THE JOURNAL OF BIOLOGICAL CHEMISTRY VOL. 282, NO. 9, pp. 6823-6832), preferably the deletion mutation is the complete deletion of LLLTDGLL (shown as SEQ ID NO: 109) and VDLLIVI (shown as SEQ ID NO: 110). By changing the nuclear localization signal and the nuclear export signal, the expression of the protein in the cytoplasm and the nucleus is regulated, thereby regulating the functional activity of the protein.

The SIRT1 sequence is a truncated version of the original sequence (UniProtKB number: Q96EB6-1), which is composed of the small molecule sirtuin-activating compounds binding domain (SBD, positions 183-229 of the original sequence), the deacetylase domain (positions 229-516 of the original sequence) and the C-terminal regulatory segment (CTR, positions 641-665 of the original sequence) of the original sequence, which are sequentially split; SIRT2-7 are the original sequences, see uniprot.org.

In certain embodiments, the SIRT1 protein, SIRT2 protein, SIRT3 protein, SIRT4 protein, SIRT5 protein, SIRT6 protein, and SIRT7 protein respectively comprise the sequence shown as any one of the following: SEQ ID NO:15-21 or SEQ ID NO:86-92, or a sequence having at least 80%, at least 85%, at least 90%, at least 91%, at least 92%, at least 93%, at least 94%, at least 95%, at least 96%, at least 97%, at least 98%, or at least 99% homology thereto, or are composed of the same, and the homologous sequence still has the function of the sequence shown as any one of SEQ ID NO: 15-21 or SEQ ID NO:86-92.

In certain embodiments, the functional mutants of the SIRT1-7 protein are mutated in the domain of the SIRT1-7 protein which is selected from the following groups: deacetylase domain, small molecule sirtuin-activating compounds binding domain (SBD), and C-terminal regulatory segment (CTR).

In certain embodiments, the SIRT1 functional mutant comprises a sequence as shown in any one of the following: SEQ ID NO:22-30 or SEQ ID NO:93-101 or a sequence having at least 80%, at least 85%, at least 90%, at least 91%, at least 92%, at least 93%, at least 94%, at least 95%, at least 96%, at least 97%, at least 98%, or at least 99% homology thereto, or is composed thereof, and the homologous sequence still has the function of the sequence shown as any one of SEQ ID NO:22-30 or SEQ ID NO: 93-101.

In some embodiments, the CD258 protein comprises a sequence as shown as SEQ ID NO:31 or SEQ ID NO:102, or a sequence having at least 80%, at least 85%, at least 90%, at least 91%, at least 92%, at least 93%, at least 94%, at least 95%, at least 96%, at least 97%, at least 98%, at least 99% homology thereto, or is composed thereof, and the homologous sequence still has the function of the sequence shown as SEQ ID NO:31 or SEQ ID NO:102. The functional mutants of the CD258 protein include membrane-bound CD258 (preferably the sequence is shown as SEQ ID NO:32 or SEQ ID NO: 103), secreted CD258 (preferably the sequence is shown as SEQ ID NO:33 or SEQ ID NO:104) and the intracellular region of CD258. In some embodiments, the membrane-bound CD258 includes the deletion or site-directed mutation of the CD258 proteolytic site. The proteolytic site is the QL sites at positions 82-83 of the sequence shown as SEQ ID NO: 102. Preferably, the deletion mutation is a complete deletion of the CD258 proteolytic site or a deletion of EQLI at positions 81-84 in the sequence as shown SEQ ID NO: 102; the substitution mutation is the substitution of Q and/or L amino acids with A, and more preferably, QL amino acids are substituted with AA. By substitution mutation or deletion on the proteolytic site, the protein is always expressed on the cell membrane in a membrane-bound form, reducing the expression of the secretory form (see uniprot.org).

In certain embodiments, the SIRT1-7 protein or its functional mutant, CD258 protein or its functional mutant comprises a sequence as shown in any of the following: SEQ ID NO: 15-33 or a sequence having at least 80%, at least 85%, at least 90%, at least 91%, at least 92%, at least 93%, at least 94%, at least 95%, at least 96%, at least 97%, at least 98%, or at least 99% homology thereto, and the homologous sequence still has the sequence function as shown in any of SEQ ID NO: 15-33, and preferably comprises a sequence as shown in any of the following: SEQ ID NO: 86-104 or a sequence having at least 80%, at least 85%, at least 90%, at least 91%, at least 92%, at least 93%, at least 94%, at least 95%, at least 96%, at least 97%, at least 98%, or at least 99% homology thereto, and the homologous sequence still has the sequence function as shown in any of SEQ ID NO: 86-104.

On the other hand, the present application provides a genetically modified immune cell, wherein the genetic modification up-regulates the expression of one or more SIRT1-7 proteins or their functional mutants, CD258 proteins or their functional mutants, and combinations thereof in the immune cell.

In certain embodiments, the up-regulation of expression is achieved by the following methods:
(1) by adding an activator that increases the expression of one or more SIRT1-7 proteins or their functional mutants, CD258 proteins or their functional mutants to the immune cell, to stimulate the immune cell itself and increase the expression of one or more SIRT1-7 proteins or their functional mutants, CD258 proteins or their functional mutants, preferably the activator is selected from the group consisting of the following: SRT2104 (CAS No. 1093403-33-8, chemical formula C₂₆H₂₄N₆O₂S₂); CAY10602 (CAS No. 374922-43-7, chemical formula C₂₂H₁₅FN₄O₂S); OSS-128167 (CAS No. 887686-02-4, chemical formula C₁₉H₁₄N₂O₆);
And/or
(2) by transfecting the immune cells with the expression vector comprising the nucleic acid encoding one or more of the SIRT1-7 proteins or their functional mutants, CD258 proteins or their functional mutants to increase the amount of one or more of the SIRT1-7 proteins or their functional mutants, CD258 proteins or their functional mutants in the immune cells; preferably, the SIRT1 or its functional mutant, SIRT2 or its functional mutant, SIRT3 or its functional mutant, SIRT4 or its functional mutant, SIRT5 or its functional mutant, SIRT6 or its functional mutant, SIRT7 protein or its functional mutant, and CD258 protein or its functional mutant are in the form of monomers or in the form of conjugates connected by connecting elements. In certain embodiments, the vector is selected from the following group: a retroviral vector, a lentiviral vector and a transposon plasmid; preferably, the vector further comprises a CAR, more preferably, the CAR is connected to a regulatory unit through a connecting element, and the regulatory unit is selected from one or more of the SIRT1-7 proteins or their functional mutants, CD258 proteins or their functional mutants, or a combination thereof.

The term “2A sequence” generally refers to a protease-independent self-cleaving amino acid sequence. The 2A sequence contributes to transcribe and produce two kinds of proteins.

In certain embodiments, the connecting element is a 2A sequence selected from the group consisting of: T2A (preferably the nucleotide sequence as shown in SEQ ID NO: 12, the amino acid sequence as shown in SEQ ID NO: 84), P2A (preferably the nucleotide sequence as shown in SEQ ID NO: 13, the amino acid sequence as shown in SEQ ID NO: 85), F2A, E2A and IRES (preferably the nucleotide sequence as shown in SEQ ID NO: 14). In certain embodiments, the connecting element comprises or consists of the sequences shown in SEQ ID NO: 84, 85 and 14.

On the other hand, the present application provides a composition comprising genetically modified immune cells.

In certain embodiments, the composition further optionally comprises a pharmaceutically acceptable carrier.

On the other hand, the present application provides the use of one or more of the SIRT1-7 proteins or their functional mutants, CD258 proteins or their functional mutants, or a combination thereof in the preparation of an agent for improving the efficacy of a drug in preventing and/or treating tumors. Preferably, the drug is a genetically modified immune cell (preferably a CAR-T cell).

On the other hand, the present application also provides a use of the genetically modified immune cell and/or the composition in the preparation of a drug, wherein the drug is used to treat and/or prevent tumors.

In certain embodiments, the tumor is selected from liver cancer, lung cancer, leukemia and mesothelioma.

In certain embodiments, the genetically modified immune cell is selected from lymphocytes. In certain embodiments, the genetically modified immune cell expresses a chimeric antigen receptor (CAR).

In certain embodiments, the method includes the steps of separating and activating the genetically modified immune cell, wherein the activation includes administering T cell culture-medium to the separated genetically modified immune cell.

In certain embodiments, the T cell culture medium is selected from one or more of the following groups: DMEM culture-medium, 1640 culture-medium, MEM culture-medium, X-VIVO culture-medium and stem cell culture-medium.

### BRIEF DESCRIPTION OF THE DRAWINGS

FIG. 1 is a schematic diagram of different CAR structures;

FIG. 2 is a diagram showing that SIRT1 protein promotes the expansion of specific CAR-T cells induced by tumor antigens;

FIG. 3 is a diagram showing that SIRT1 protein inhibits the expression of immune negative regulatory proteins;

FIG. 4 is a diagram showing that SIRT1 protein promotes the proliferation of memory T cells in CD3 cells;

FIG. 5 is a diagram showing that SIRT1 protein promotes the proliferation of memory T cells in CD4 and CD8 cells;

FIGS. 6-8 are diagrams showing that functional mutants of SIRT1 protein and their combinations inhibit the expression of negative immune regulatory proteins;

FIGS. 9-10 are diagrams showing that functional mutants of SIRT1 protein and their combinations promote the proliferation of memory T cells in CD3 cells;

FIG. 11 shows that the functional mutants of SIRT1 protein and their combinations promote the proliferation of memory T cells in CD4 cells;

FIG. 12 shows that the functional mutants of SIRT1 protein and their combinations promote the proliferation of memory T cells in CD8 cells;

FIG. 13 shows that the functional mutants of SIRT1 protein and their combinations promote the proliferation of T cells;

FIG. 14 shows that the functional mutants of SIRT1 protein and their combinations promote the resistance of T cells to immune suppressive micro-environment;

FIG. 15 shows that CD258 protein and the functional mutant thereof enhance the killing ability of CAR-T cells against tumor cells;

FIG. 16 shows that the combination of CD258 protein and SIRT1 protein promotes the expansion of specific CAR-T cells induced by tumor antigens;

FIG. 17 shows that the combination of CD258 protein and SIRT1 protein promotes the proliferation of memory T cells in CD3 cells (GPC3);

FIG. 18 shows that the combination of CD258 protein and SIRT1 protein promotes the proliferation of memory T cells in CD4 and CD8 cells (GPC3);

FIG. 19 shows that the combination of CD258 protein and SIRT1 protein inhibits the expression of immune negative regulatory proteins (CD19);

FIG. 20 shows that the combination of CD258 protein and SIRT1 protein promotes the proliferation of memory T cells in CD8 cells (CD19);

FIG. 21 shows that the combination of CD258 protein and SIRT1 protein promotes the proliferation of memory T cells in CD3 and CD4 cells Proliferation (CD19);

FIG. 22 shows that the combination of CD258 protein and SIRT1 protein inhibits the expression of negative immune regulatory proteins (MSLN);

FIG. 23 shows that the combination of CD258 protein and SIRT1 protein promotes the proliferation of memory T cells in CD3 cells (MSLN);

FIG. 24 shows that the combination of CD258 protein and SIRT1 protein enhances the killing ability of CAR-T cells against tumor cells;

FIG. 25 shows that the combination of CD258 protein and SIRT1 protein enhances the anti-tumor effects of CAR-T cells;

FIG. 26 shows that the combination of CD258 protein and SIRT1 protein promotes the proliferation of granulocytes and monocytes in vivo;

FIG. 27 shows that the combination of CD258 protein and SIRT1 protein promotes the proliferation of CAR-T cells in vivo and the release of Th1 cytokines.

### DETAILED DESCRIPTION OF THE EMBODIMENTS

The present invention is further described below by examples. However, it should be understood that the examples are for illustrative purposes only and are not intended to limit the scope and spirit of the present invention.

Those skilled in the art can easily gain insight into other aspects and advantages of the present invention from the detailed description below. Only exemplary embodiments of the present invention are shown and described in the detailed description below. As those skilled in the art will recognize, the content of the present invention enables those skilled in the art to modify the disclosed specific embodiments without departing from the spirit and scope of the invention involved in this application.

Accordingly, the descriptions in the drawings and specifications of this application are merely exemplary and not restrictive.

### Example 1 Construction of Lentivirus Vector

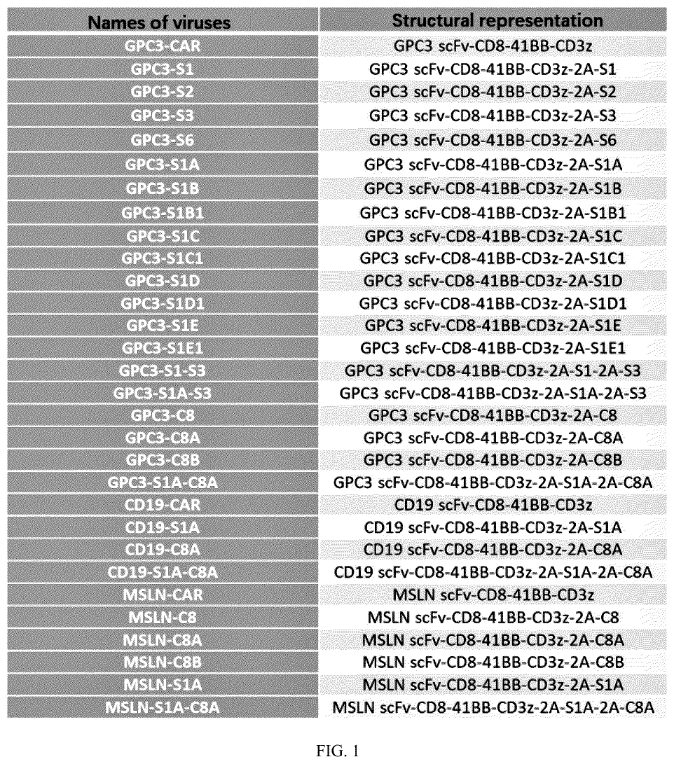

The CAR-T targeting GPC3, CD19, and Mesothelin (MSLN) is used as an example, artificially synthesize a fragment containing the CAR structure and construct it into a lentivirus vector (LV100A, System Biosciences), then transfect in accordance with the method described in its specification to obtain the lentivirus comprising GPC3-CAR, GPC3-S1, GPC3-S2, GPC3-S3, GPC3-S6, GPC3-S1A, GPC3-S1B, GPC3-S1B1, GPC3-S1C, GPC3-S1C1, GPC3-S1D, GPC3-S1D1, GPC3-S1E, GPC3-S1E1, GPC3-S1-S3, GPC3-S1A-S3, GPC3-C8, GPC3-C8A, GPC3-C8B, GPC3-S1A-C8A, CD19-CAR, CD19-S1A, CD19-C8A, CD19-S1A-C8A, MSLN-CAR, MSLN-C8, MSLN-C8A, MSLN-C8B, MSLN-S1A, MSLN-S1A-C8A. The schematic diagram of each CAR structure is shown in FIG. 1.

GPC3-CAR is synthesized by splicing SEQ ID NO:1, SEQ ID NO:2, SEQ ID NO:7, SEQ ID NO:8, SEQ ID NO: 10, and SEQ ID NO: 11 in sequence.

GPC3-S1 was synthesized by splicing SEQ ID NO:1, SEQ ID NO:2, SEQ ID NO:7, SEQ ID NO:8, SEQ ID NO:10, SEQ ID NO: 11, SEQ ID NO:12, and SEQ ID NO: 15 in sequence.
GPC3-S2 was synthesized by splicing SEQ ID NO:1, SEQ ID NO:2, SEQ ID NO:7, SEQ ID NO:8, SEQ ID NO: 10, SEQ ID NO: 11, SEQ ID NO:12, and SEQ ID NO:16 in sequence.
GPC3-S3 was synthesized by splicing SEQ ID NO:1, SEQ ID NO:2, SEQ ID NO:7, SEQ ID NO:8, SEQ ID NO:10, SEQ ID NO:11, SEQ ID NO:12, and SEQ ID NO:17 in sequence.
GPC3-S6 was synthesized by splicing SEQ ID NO:1, SEQ ID NO:2, SEQ ID NO:7, SEQ ID NO:8, SEQ ID NO:10, SEQ ID NO:11, SEQ ID NO:12, and SEQ ID NO:20 in sequence.
GPC3-S1A was synthesized by splicing SEQ ID NO:1, SEQ ID NO:2, SEQ ID NO:7, SEQ ID NO:8, SEQ ID NO:10, SEQ ID NO:11, SEQ ID NO:12, and SEQ ID NO:22 in sequence.
GPC3-S1B consists of SEQ ID NO:1, SEQ ID NO:2, SEQ ID NO:7, SEQ ID NO:8, SEQ ID NO:10, SEQ ID NO:11, SEQ ID NO:12, SEQ ID NO: 23 are spliced and synthesized in sequence.
GPC3-S1B1 consists of SEQ ID NO:1, SEQ ID NO:2, SEQ ID NO:7, SEQ ID NO:8, SEQ ID NO: 10, SEQ ID NO:11, SEQ ID NO: 12. SEQ ID NO: 24 was spliced and synthesized in sequence.
GPC3-SIC consists of SEQ ID NO:1, SEQ ID NO:2, SEQ ID NO:7, SEQ ID NO:8, SEQ ID NO: 10, SEQ ID NO:11, SEQ ID NO: 12. SEQ ID NO: 25 was spliced and synthesized in sequence.
GPC3-S1C1 consists of SEQ ID NO:1, SEQ ID NO:2, SEQ ID NO:7, SEQ ID NO:8, SEQ ID NO: 10, SEQ ID NO:11, SEQ ID NO: 12. SEQ ID NO: 26 was spliced and synthesized in sequence.
GPC3-S1D consists of SEQ ID NO:1, SEQ ID NO:2, SEQ ID NO:7, SEQ ID NO:8, SEQ ID NO:10, SEQ ID NO:11, SEQ ID NO: 12. SEQ ID NO: 27 was spliced and synthesized in sequence.
GPC3-S1D1 consists of SEQ ID NO:1, SEQ ID NO:2, SEQ ID NO:7, SEQ ID NO:8, SEQ ID NO:10, SEQ ID NO:11, SEQ ID NO: 12. SEQ ID NO: 28 was spliced and synthesized in sequence.
GPC3-S1E consists of SEQ ID NO:1, SEQ ID NO:2, SEQ ID NO:7, SEQ ID NO:8, SEQ ID NO: 10, SEQ ID NO:11, SEQ ID NO: 12. SEQ ID NO: 29 was spliced and synthesized in sequence.
GPC3-S1E1 consists of SEQ ID NO:1, SEQ ID NO:2, SEQ ID NO:7, SEQ ID NO:8, SEQ ID NO: 10, SEQ ID NO:11, SEQ ID NO: 12. SEQ ID NO: 30 was spliced and synthesized in sequence.
GPC3-S1-S3 consists of SEQ ID NO:1, SEQ ID NO:2, SEQ ID NO:7, SEQ ID NO:8, SEQ ID NO:10, SEQ ID NO:11, SEQ ID NO: 12, SEQ ID NO:15, SEQ ID NO:13, and SEQ ID NO: 17 were spliced and synthesized in sequence.
GPC3-S1A-S3 consists of SEQ ID NO:1, SEQ ID NO:2, SEQ ID NO:7, SEQ ID NO:8, SEQ ID NO: 10, SEQ ID NO:11, SEQ ID NO: 12, SEQ ID NO:22, SEQ ID NO: 13, and SEQ ID NO: 17 were spliced and synthesized in sequence.
GPC3-C8 consists of SEQ ID NO:1, SEQ ID NO:2, SEQ ID NO:7, SEQ ID NO:8, SEQ ID NO:10, SEQ ID NO:11, SEQ ID NO: 12. SEQ ID NO:31 was spliced and synthesized in sequence.
GPC3-C8A consists of SEQ ID NO:1, SEQ ID NO:2, SEQ ID NO:7, SEQ ID NO:8, SEQ ID NO: 10, SEQ ID NO:11, SEQ ID NO: 12. SEQ ID NO: 32 was spliced and synthesized in sequence.
GPC3-C8B consists of SEQ ID NO:1, SEQ ID NO:2, SEQ ID NO:7, SEQ ID NO:8, SEQ ID NO:10, SEQ ID NO:11, SEQ ID NO: 12. SEQ ID NO: 33 was spliced and synthesized in sequence.
GPC3-S1A-C8A consists of SEQ ID NO:1, SEQ ID NO:2, SEQ ID NO:7, SEQ ID NO:8, SEQ ID NO: 10, SEQ ID NO:11, SEQ ID NO: 12, SEQ ID NO:22, SEQ ID NO: 13, and SEQ ID NO:32 were spliced and synthesized in sequence.
CD19-CAR is synthesized by splicing SEQ ID NO:1, SEQ ID NO:3, SEQ ID NO:7, SEQ ID NO:8, SEQ ID NO: 10 and SEQ ID NO:11 in sequence.
CD19-S1A is composed of SEQ ID NO:1, SEQ ID NO:3, SEQ ID NO:7, SEQ ID NO:8, SEQ ID NO: 10, SEQ ID NO:11, SEQ ID NO:12, SEQ ID NO: 22 are spliced and synthesized in sequence.
CD19-C8A consists of SEQ ID NO:1, SEQ ID NO:3, SEQ ID NO:7, SEQ ID NO:8, SEQ ID NO: 10, SEQ ID NO:11, SEQ ID NO: 12. SEQ ID NO: 32 was spliced and synthesized in sequence.
CD19-S1A-C8A consists of SEQ ID NO:1, SEQ ID NO:3, SEQ ID NO:7, SEQ ID NO:8, SEQ ID NO: 10, SEQ ID NO:11, SEQ ID NO: 12, SEQ ID NO:22, SEQ ID NO: 13, and SEQ ID NO:32 were spliced and synthesized in sequence.
MSLN-CAR is synthesized by splicing SEQ ID NO:1, SEQ ID NO:5, SEQ ID NO:7, SEQ ID NO:8, SEQ ID NO: 10, and SEQ ID NO: 11 in sequence.
MSLN-S1A consists of SEQ ID NO:1, SEQ ID NO:5, SEQ ID NO:7, SEQ ID NO:8, SEQ ID NO:10, SEQ ID NO:11, SEQ ID NO: 12. SEQ ID NO: 22 was spliced and synthesized in sequence.
MSLN-C8 consists of SEQ ID NO:1, SEQ ID NO:5, SEQ ID NO:7, SEQ ID NO:8, SEQ ID NO:10, SEQ ID NO:11, SEQ ID NO: 12. SEQ ID NO:31 was spliced and synthesized in sequence.
MSLN-C8A consists of SEQ ID NO:1, SEQ ID NO:5, SEQ ID NO:7, SEQ ID NO:8, SEQ ID NO: 10, SEQ ID NO:11, SEQ ID NO: 12. SEQ ID NO: 32 was spliced and synthesized in sequence.
MSLN-C8B consists of SEQ ID NO:1, SEQ ID NO:5, SEQ ID NO:7, SEQ ID NO:8, SEQ ID NO:10, SEQ ID NO:11, SEQ ID NO: 12. SEQ ID NO: 33 was spliced and synthesized in sequence.
MSLN-S1A-C8A consists of SEQ ID NO:1, SEQ ID NO:5, SEQ ID NO:7, SEQ ID NO:8, SEQ ID NO: 10, SEQ ID NO:11, SEQ ID NO:12, SEQ ID NO:22, SEQ ID NO: 13, and SEQ ID NO:32 were sequentially spliced and synthesized.

### Example 2: Infecting T Cells with Lentivirus

Infection experiment was performed according to the conventional approach known by those skilled in the art. The infection steps are briefly described as follows:
1. Obtain peripheral blood mononuclear lymphocytes (PBMCs), obtain more than 1×10⁷ cells through a blood apheresis system.
2. Treat the cell culture dish with anti-human CD3/CD28 antibodies.
Dilute the anti-human CD3 and anti-human CD28 antibodies (purchased from Shanghai Jinan Technology Co., Ltd.) with PBS to a final concentration of 1 ug/ml, add the diluted antibody mixture to the cell culture dish to cover the entire dish, and incubate at room temperature for 2 hours. 2 hours later, wash once with PBS and set aside.
3. Activate T cells
Resuspend the separated PBMC with T lymphocyte culture medium (Xvivo15 medium+5% FBS+100 U/mlIL2+20 ng/mlIL21+20 ng/mlIL7) to a final concentration of 1×10⁶ cells/ml, and cultured in the culture dish which are treated in step 2 at 37° C.+5% CO₂, and culture for 24 hours.
4. Infect the activated T cells
1) Prepare the infection reagent.
Take a certain amount of T cell culture medium and add synperonic F108 (Sigma) at a final concentration of 1 mg/ml, mix well, and heat to 37° C. in a water bath for later use.
2) treating the culture plates.
Take 1 mg/ml anti-human CD3 antibody and 0.5 mg/ml anti-human CD28 antibody and dilute them in an appropriate amount of PBS buffer at a volume ratio of 1:1000, and take retronectin (1 mg/ml, Takara) reagent, dilute it into the PBS buffer at a volume ratio of 1:40, mix well and evenly spread it on the cell dish, incubate at room temperature for 2 hours. After 2 hours, wash it with PBS and set aside.
3) Infecting T cells with Lentivirus.
Dilute the activated T cells with the infection reagent prepared in 1), add lentivirus at an MOI of 3, and mix well. Evenly spread in the culture dish treated in 2).
After infecting, monitor the cell density and maintain the cell density at 1×10⁶ cells/ml. Generally, the cell density can be expanded 30-1000 times in 14 days.

### Example 3: SIRT1 Protein Promotes Tumor Antigen-Induced Expansion of Specific CAR-T Cells

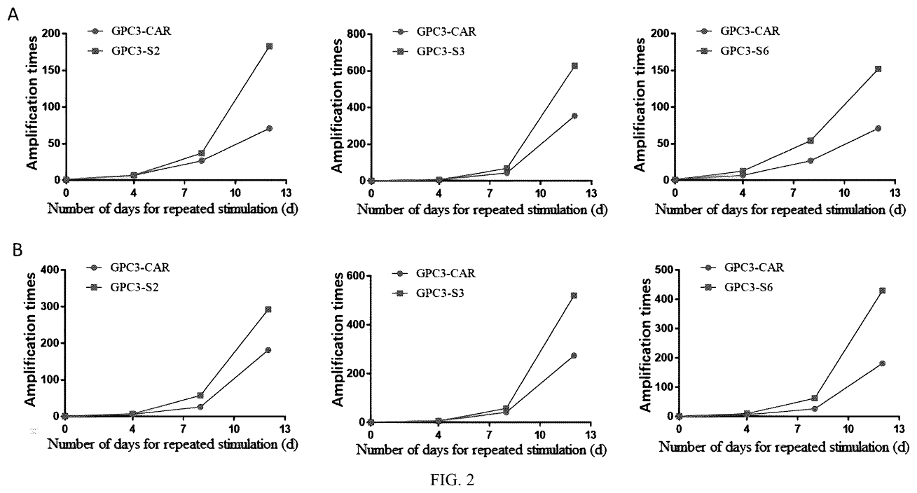

T cells expressing GPC3-CAR, GPC3-S2, GPC3-S3 or GPC3-S6 were co-cultured with irradiated (X-RAD cell irradiator, irradiation dose of 30Gy) HepG2 cells (purchased from the Cell Bank of Chinese Academy of Sciences) at a cell number ratio of 1:1 using Xvivo15 medium, and irradiated HepG2 was re-added every 4 days for stimulation, and the stimulation was repeated 3 times. Each time, the cells were counted by trypan blue staining, and the cell proliferation is shown in FIG. 2A. The results showed that the expansion multiples of T cells expressing GPC3-S2, GPC3-S3 or GPC3-S6 were much higher than those of T cells expressing GPC3-CAR in the control group.

Similarly, T cells expressing GPC3-CAR, GPC3-S2, GPC3-S3 or GPC3-S6 were co-cultured with irradiated (X-RAD cell irradiator, irradiation dose of 30Gy) Huh7 cells (purchased from the cell bank of the Chinese Academy of Sciences) at a cell number ratio of 1:1 using Xvivo15 medium, and irradiated Huh7 was re-added every 4 days for stimulation, and the stimulation was repeated 3 times. Each time, the cells were counted by trypan blue staining, and the cell proliferation is shown in FIG. 2B. The results also showed that the expansion multiples of T cells expressing GPC3-S2, GPC3-S3 or GPC3-S6 were much higher than those of T cells expressing GPC3-CAR in the control group.

### Example 4: SIRT1 Protein Inhibits the Expression of Immune Negative Regulatory Proteins

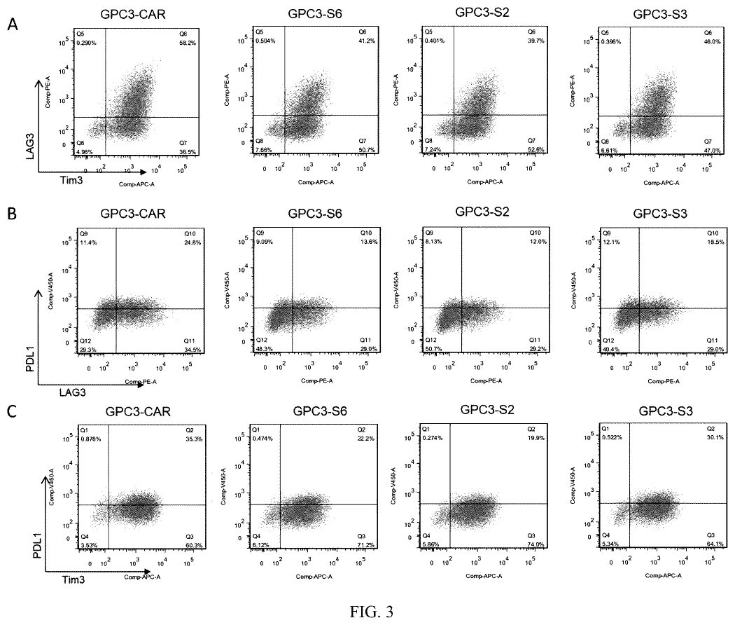

The T cells expressing GPC3-CAR, GPC3-S2, GPC3-S3 or GPC3-S6 obtained in Example 2 were cultured in vitro at 37° C. and 5% CO₂ in a cell culture incubator for 9 or 12 days. The expression of CD3, CD4, CD8, PD1, PDL1, TIM3 and LAG3 proteins in T cells was detected by BD flow cytometer. The results are shown in FIG. 3. In T cells expressing GPC3-S2, GPC3-S3 or GPC3-S6, the proportion of cells expressing two negative regulatory proteins at the same time was significantly lower than that of the GPC3-CAR control group.

### Example 5: SIRT1 Protein Promotes the Proliferation of Memory T Cells in CD3 Cells

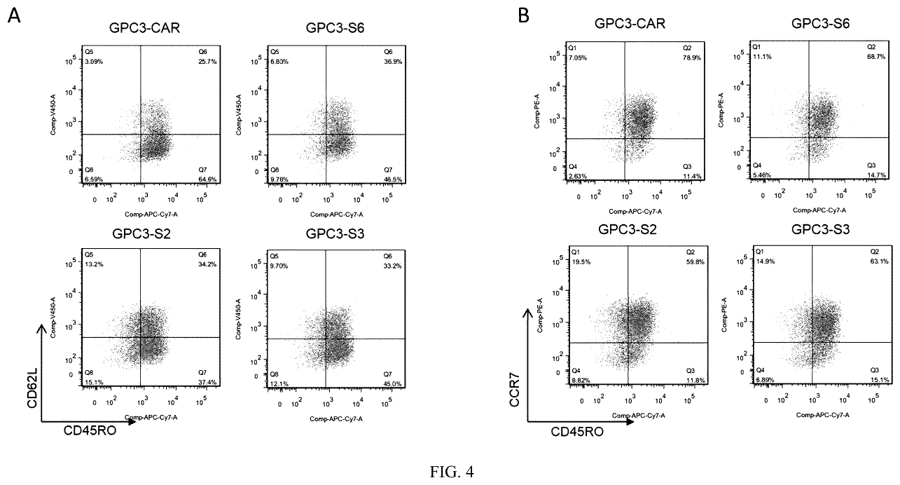

The T cells expressing GPC3-CAR, GPC3-S2, GPC3-S3 or GPC3-S6 obtained in Example 2 were cultured in vitro at 37° C. and 5% CO₂ in a cell culture incubator for 9 or 12 days. The expression of CD3, CD45RO, CD45RA, CD62L, CCR7, CD95, CD122, CD127, CD27 and CD28 proteins in T cells was detected by BD flow cytometer. As shown in FIG. 4, the proportion of memory stem cell-like T cells (TSCM) in CD3 cells expressing GPC3-S2, GPC3-S3 or GPC3-S6 in the total cells was significantly higher than that in the GPC3-CAR control group.

### Example 6: SIRT1 Protein Promotes the Proliferation of Memory T Cells in CD4 and CD8 Cells

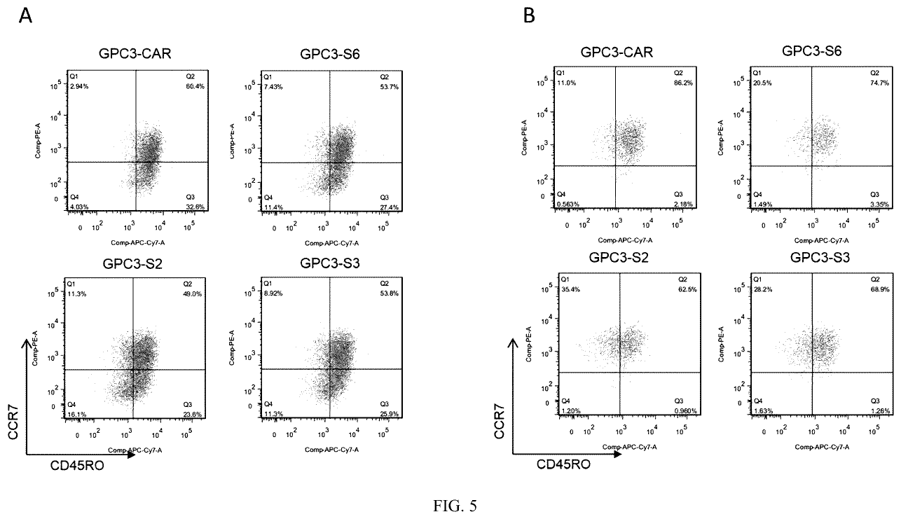

The T cells expressing GPC3-CAR, GPC3-S2, GPC3-S3 or GPC3-S6 obtained in Example 2 were cultured in vitro in a cell culture incubator at 37° C. and 5% CO₂ for 9 or 12 days. The expression of CD4, CD45RO, CD45RA, CD62L, CCR7, CD95, CD122, CD127, CD27 and CD28 proteins in T cells was detected by BD flow cytometer. As shown in FIG. 5A, the proportion of memory stem cell-like T cells (TSCM) in CD4 cells expressing GPC3-S2, GPC3-S3 or GPC3-S6 in the total cells was significantly higher than that in the GPC3-CAR control group.

Similarly, the T cells expressing GPC3-CAR, GPC3-S2, GPC3-S3 or GPC3-S6 obtained in Example 2 were cultured in vitro at 37° C. and 5% CO₂ in a cell culture incubator for 9 or 12 days. The expression of CD8, CD45RO, CD45RA, CD62L, CCR7, CD95, CD122, CD127, CD27, and CD28 proteins in T cells was detected by BD flow cytometer. As shown in FIG. 5B, the proportion of memory stem cell-like T cells (TSCM) in CD8 cells expressing GPC3-S2, GPC3-S3 or GPC3-S6 in the total cells was significantly higher than that in the GPC3-CAR control group.

### Example 7: The Functional Mutants of SIRT1 Protein and their Combinations Inhibit the Expression of Immune Negative Regulatory Proteins

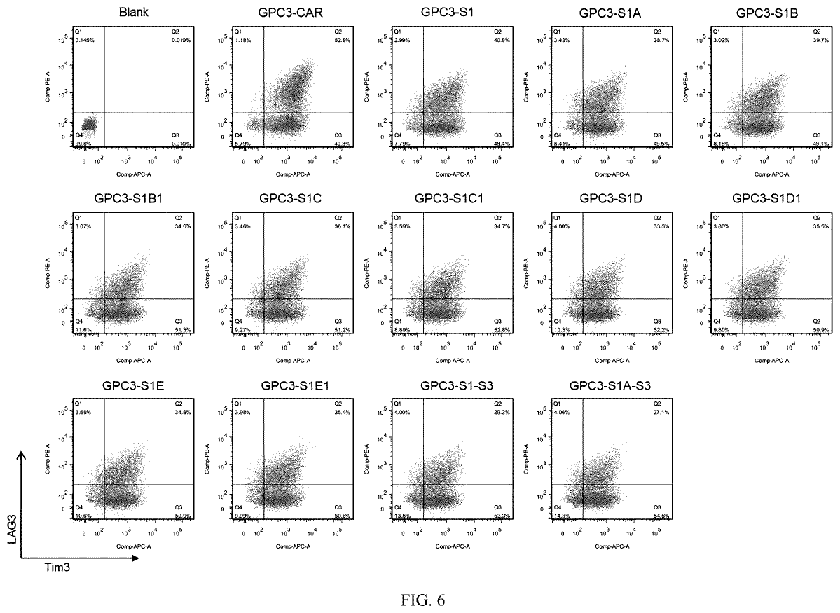

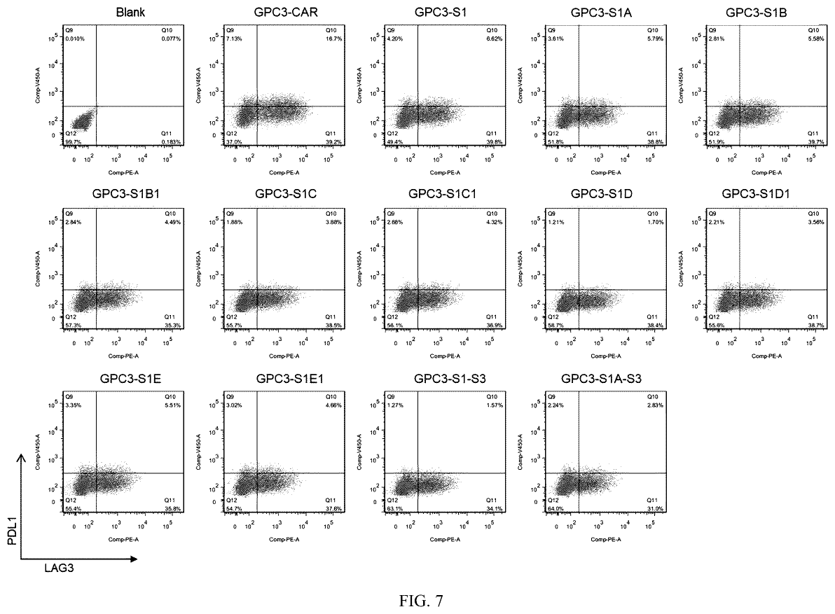

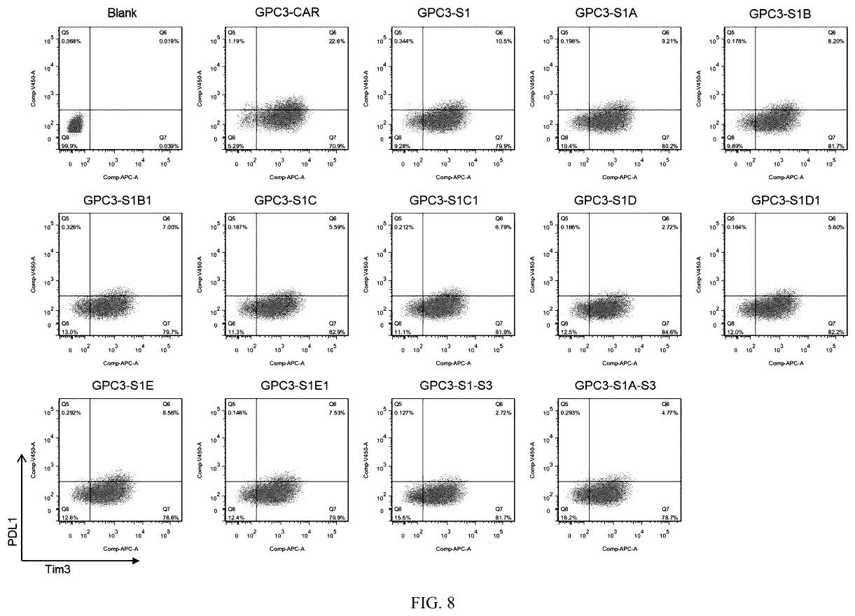

The T cells expressing GPC3-CAR, GPC3-S1, GPC3-S1A, GPC3-SIB, GPC3-S1B1, GPC3-SIC, GPC3-S1C1, GPC3-S1D, GPC3-S1D1, GPC3-S1E, GPC3-S1E1, GPC3-S1-S3 and GPC3-S1A-S3 respectively obtained in Example 2 were cultured in vitro in a 37° C., 5% CO₂ cell culture incubator for 9 days or 12 days. The expression of CD3, CD4, CD8, PD1, PDL1, TIM3, and LAG3 proteins in T cells was detected by BD flow cytometer. The results are shown in FIGS. 6-8. In T cells expressing GPC3-S1, GPC3-S1A, GPC3-S1B, GPC3-S1B1, GPC3-S1C, GPC3-S1C1, GPC3-S1D, GPC3-S1D1, GPC3-S1E, GPC3-S1E1, GPC3-S1-S3 or GPC3-S1A-S3, the proportion of cells expressing two negative regulatory proteins at the same time was significantly lower than that of the GPC3-CAR control group.

### Example 8: The Functional Mutants of SIRT1 Protein and their Combinations Promote the Proliferation of Memory T Cells in CD3 Cells

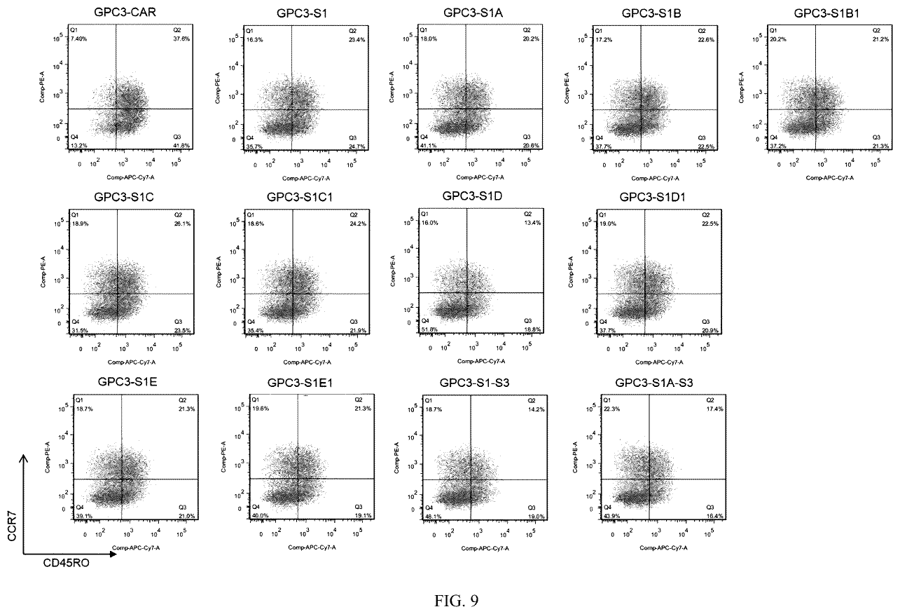

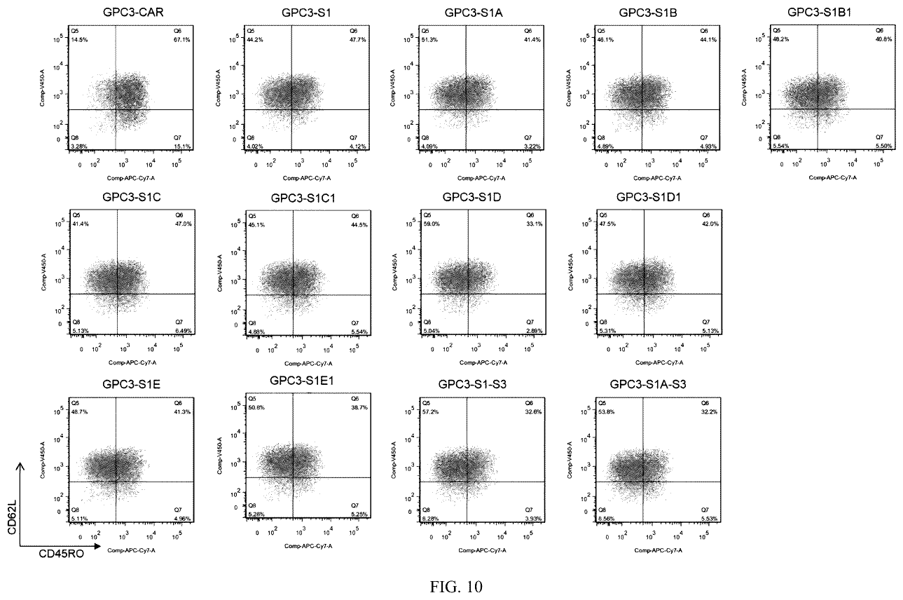

The T cells expressing GPC3-CAR, GPC3-S1, GPC3-S1A, GPC3-SIB, GPC3-S1B1, GPC3-SIC, GPC3-S1C1, GPC3-S1D, GPC3-S1D1, GPC3-S1E, GPC3-S1E1, GPC3-S1-S3 and GPC3-S1A-S3 obtained in Example 2 were cultured in vitro in a 37° C., 5% CO₂ cell culture incubator for 9 days or 12 days. The expression of CD3, CD45RO, CD45RA, CD62L, CCR7, CD95, CD122, CD127, CD27 and CD28 proteins in T cells was detected by BD flow cytometer. As shown in FIGS. 9-10, the proportion of memory stem cell-like T cells (TSCM) in CD3 cells expressing GPC3-S1, GPC3-S1A, GPC3-S1B, GPC3-S1B1, GPC3-S1C, GPC3-S1C1, GPC3-S1D, GPC3-S1D1, GPC3-S1E, GPC3-S1E1, GPC3-S1-S3 and GPC3-S1A-S3 was significantly higher than that in the GPC3-CAR control group.

### Example 9: The Functional Mutants of SIRT1 Protein and Combinations Thereof Promote the Proliferation of Memory T Cells in CD4 Cells

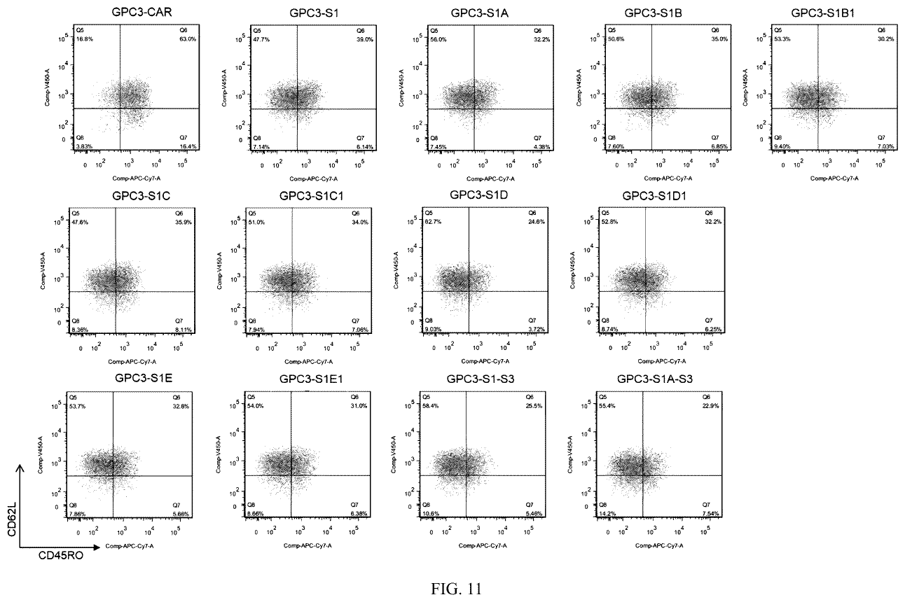

The T cells expressing GPC3-CAR, GPC3-S1, GPC3-S1A, GPC3-SIB, GPC3-S1B1, GPC3-SIC, GPC3-S1C1, GPC3-S1D, GPC3-S1D1, GPC3-S1E, GPC3-S1E1, GPC3-S1-S3 and GPC3-S1A-S3 respectively obtained in Example 2 were cultured in vitro in a 37° C., 5% CO₂ cell culture incubator for 9 days or 12 days. The expression of CD4, CD45RO, CD45RA, CD62L, CCR7, CD95, CD122, CD127, CD27 and CD28 proteins in T cells was detected by BD flow cytometer. As shown in FIG. 11, the proportion of memory stem cell-like T cells (TSCM) in CD4 cells expressing GPC3-S1, GPC3-S1A, GPC3-S1B, GPC3-S1B1, GPC3-S1C, GPC3-S1C1, GPC3-S1D, GPC3-S1D1, GPC3-S1E, GPC3-S1E1, GPC3-S1-S3 and GPC3-S1A-S3 was significantly higher than that in the GPC3-CAR control group.

### Example 10: The Functional Mutants of SIRT1 Protein and Combinations Thereof Promote the Proliferation of Memory T Cells in CD8 Cells

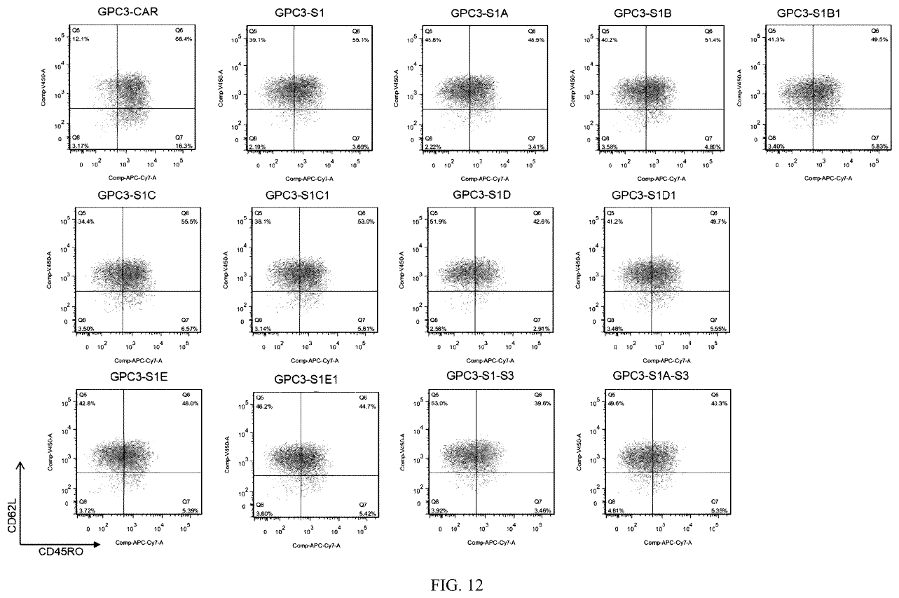

The T cells expressing GPC3-CAR, GPC3-S1, GPC3-S1A, GPC3-SIB, GPC3-S1B1, GPC3-SIC, GPC3-S1C1, GPC3-S1D, GPC3-S1D1, GPC3-S1E, GPC3-S1E1, GPC3-S1-S3 and GPC3-S1A-S3 respectively obtained in Example 2 were cultured in vitro in a 37° C., 5% CO₂ cell culture incubator for 9 days or 12 days. The expression of CD8, CD45RO, CD45RA, CD62L, CCR7, CD95, CD122, CD127, CD27 and CD28 proteins in T cells was detected by BD flow cytometer. As shown in FIG. 12, the proportion of memory stem cell-like T cells (TSCM) in the CD8 cells expressing GPC3-S1, GPC3-S1A, GPC3-S1B, GPC3-S1B1, GPC3-S1C, GPC3-S1C1, GPC3-S1D, GPC3-S1D1, GPC3-S1E, GPC3-S1E1, GPC3-S1-S3 and GPC3-S1A-S3 was significantly higher than that in the GPC3-CAR control group.

### Example 11: The Functional Mutants of SIRT1 Protein and Combinations Thereof Promote the Proliferation of T Cells

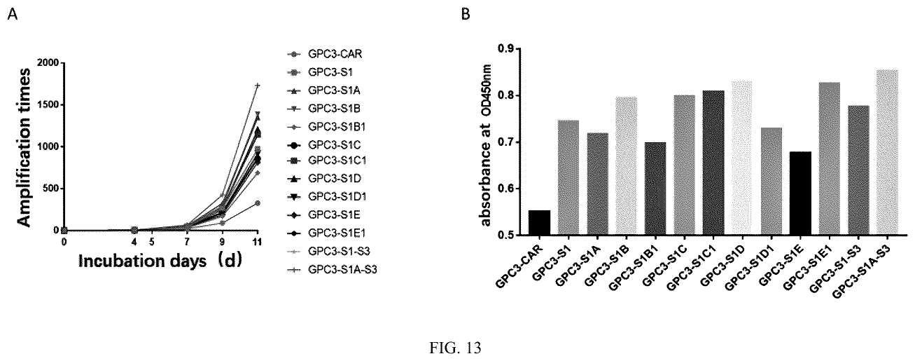

The T cells expressing GPC3-CAR, GPC3-S1, GPC3-S1A, GPC3-SIB, GPC3-S1B1, GPC3-SIC, GPC3-S1C1, GPC3-S1D, GPC3-S1D1, GPC3-S1E, GPC3-S1E1, GPC3-S1-S3, and GPC3-S1A-S3 respectively obtained in Example 2 were cultured in vitro in a 37° C., 5% CO₂ cell culture incubator for 11 days. The cells were counted using trypan blue staining and passaged every 2-3 days. The cell proliferation is shown in FIG. 13A. The results showed that the expansion multiples of T cells expressing GPC3-S1, GPC3-S1A, GPC3-SIB, GPC3-S1B1, GPC3-S1C, GPC3-S1C1, GPC3-S1D, GPC3-S1D1, GPC3-S1E, GPC3-S1E1, GPC3-S1-S3 and GPC3-S1A-S3 were much higher than those of T cells expressing GPC3-CAR in the control group.

Similarly, the T cells expressing GPC3-CAR, GPC3-S1, GPC3-S1A, GPC3-SIB, GPC3-S1B1, GPC3-S1C, GPC3-S1C1, GPC3-S1D, GPC3-S1D1, GPC3-S1E, GPC3-S1E1, GPC3-S1-S3 and GPC3-S1A-S3 obtained in Example 2 were cultured in vitro in a 37° C., 5% CO₂ cell culture incubator and cultured for 7 days. On the 7th day, the cells were taken and incubated with Xvivo15 culture medium for 24 hours, and CCK8 (MCE Company) was used to detect the proliferation effect of the T cell. As shown in FIG. 13B, the proliferation effect of T cells expressing GPC3-S1, GPC3-S1A, GPC3-S1B, GPC3-S1B1, GPC3-S1C, GPC3-S1C1, GPC3-S1D, GPC3-S1D1, GPC3-S1E, GPC3-S1E1, GPC3-S1-S3 and GPC3-S1A-S3 was also much higher than that of T cells expressing GPC3-CAR in the control group.

### Example 12: The Functional Mutants of SIRT1 Protein and Combinations Thereof Promote the Resistance of T Cells to Immunosuppressive Microenvironment

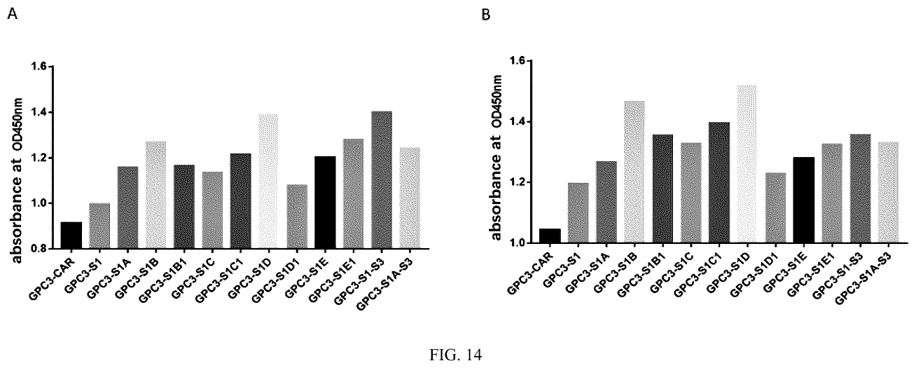

The T cells expressing GPC3-CAR, GPC3-S1, GPC3-S1A, GPC3-SIB, GPC3-S1B1, GPC3-SIC, GPC3-S1C1, GPC3-S1D, GPC3-S1D1, GPC3-S1E, GPC3-S1E1, GPC3-S1-S3 and GPC3-S1A-S3 respectively obtained in Example 2 were cultured in vitro in a 37° C., 5% CO₂ cell culture incubator for 8 days. On the 8th day, the cells were taken and incubated with 1640 glucose-free and serum-free culture medium (Gibco) for 24 hours, and CCK8 (MCE) was used to detect the proliferation effect of the T cell. The results are shown in FIG. 14A. The proliferation effect of T cells expressing GPC3-S1, GPC3-S1A, GPC3-S1B, GPC3-S1B1, GPC3-S1C, GPC3-S1C1, GPC3-S1D, GPC3-S1D1, GPC3-S1E, GPC3-S1E1, GPC3-S1-S3 and GPC3-S1A-S3 was much higher than that of T cells expressing GPC3-CAR in the control group.

Similarly, the T cells expressing GPC3-CAR, GPC3-S1, GPC3-S1A, GPC3-S1B, GPC3-S1B1, GPC3-SIC, GPC3-S1C1, GPC3-S1D, GPC3-S1D1, GPC3-S1E, GPC3-S1E1, GPC3-S1-S3 and GPC3-S1A-S3 obtained in Example 2 were cultured in vitro in a 37° C., 5% CO₂ cell culture incubator and cultured for 8 days. On the 8th day, the cells were taken and incubated with 1640 glucose-free and serum-free culture medium (Gibco) and 20 mM lactic acid (PH value dropped from normal 7.4 to 6.5, Sigma) for 24 hours, and CCK8 (MCE) was used to detect the T cell proliferation effect. As shown in FIG. 14B, the proliferation effect of T cells expressing GPC3-S1, GPC3-S1A, GPC3-SIB, GPC3-S1B1, GPC3-SIC, GPC3-S1C1, GPC3-S1D, GPC3-S1D1, GPC3-S1E, GPC3-S1E1, GPC3-S1-S3, and GPC3-S1A-S3 was also much higher than that of T cells expressing GPC3-CAR in the control group.

### Example 13: CD258 Protein and the Functional Mutant Thereof Enhance the Killing Ability of CAR-T Cells Against Tumor Cells

T cells expressing GPC3-CAR, GPC3-C8, GPC3-C8A or GPC3-C8B were co-cultured with Huh7 cells (purchased from the cell bank of the Chinese Academy of Sciences) at a cell number ratio of 1:15 using Xvivo15 medium for 7 days, and crystal violet staining (MCE) was used to detect the killing effect of CAR-T cells on tumor cells. The results are shown in FIG. 15A. The killing effect of T cells expressing GPC3-C8 or GPC3-C8A on tumor cells was much higher than that of T cells expressing GPC3-CAR in the control group, among which T cells expressing GPC3-C8A had a more superior killing effect than T cells expressing GPC3-C8.

Similarly, T cells expressing GPC3-CAR, GPC3-C8, GPC3-C8A or GPC3-C8B were co-cultured with Huh7 cells (purchased from the Cell Bank of Chinese Academy of Sciences) at a cell number ratio of 1:15 using 1640 glucose-free serum-free (Gibco) and 20 mM lactate (PH value dropped from normal 7.4 to 6.5, Sigma) medium for 7 days, and crystal violet staining (MCE) was used to detect the killing effect of CAR-T cells on tumor cells. The results are shown in FIG. 15B. The killing effect of T cells expressing GPC3-C8 or GPC3-C8A on tumor cells is also significantly higher than that of T cells expressing GPC3-CAR in the control group. T cells expressing GPC3-C8A also have a superior killing effect compared to T cells expressing GPC3-C8.

### Example 14: The Combination of CD258 Protein and SIRT1 Protein Promotes Tumor Antigen-Induced Specific CAR-T Cell Expansion

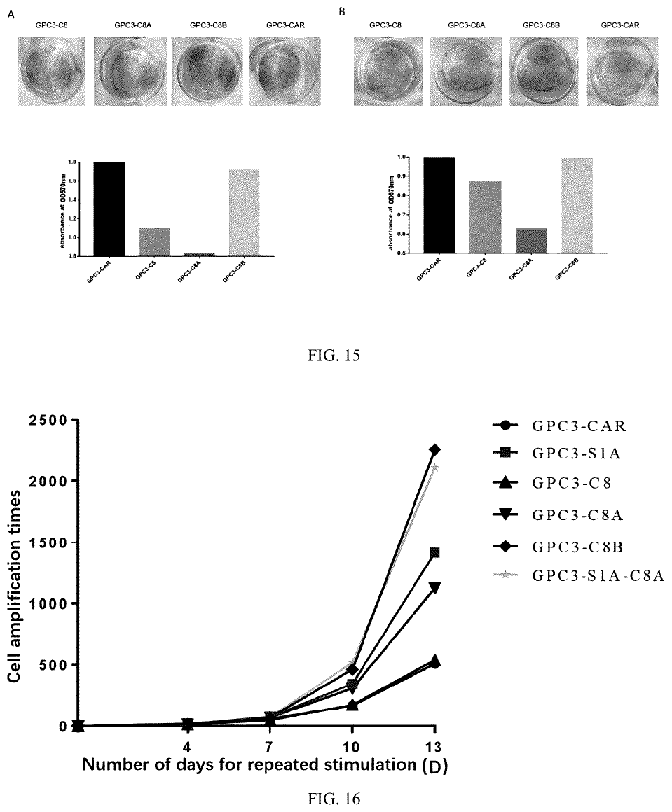

T cells expressing GPC3-CAR, GPC3-S1A, GPC3-C8, GPC3-C8A, GPC3-C8B or GPC3-S1A-C8A were co-cultured with irradiated (X-RAD cell irradiator, irradiation dose of 30Gy) Huh7 cells (purchased from the Cell Bank of Chinese Academy of Sciences) at a 1:1 cell number ratio using Xvivo15 medium, and irradiated Huh7 was re-added every 3-4 days for stimulation, and the stimulation was repeated 4 times. Each time, the cells were counted by trypan blue staining, and the cell proliferation is shown in FIG. 16. The results showed that the expansion multiples of T cells expressing GPC3-S1A, GPC3-C8A, GPC3-C8B or GPC3-S1A-C8A were much higher than those of T cells expressing GPC3-CAR in the control group.

GPC3-S1A-C8A is consisted of GPC3, S1A and C8A which are coupled sequentially through the 2A sequence.

### Example 15: The Combination of CD258 Protein and SIRT1 Protein Promotes the Proliferation of Memory T Cells in CD3 Cells (GPC3)

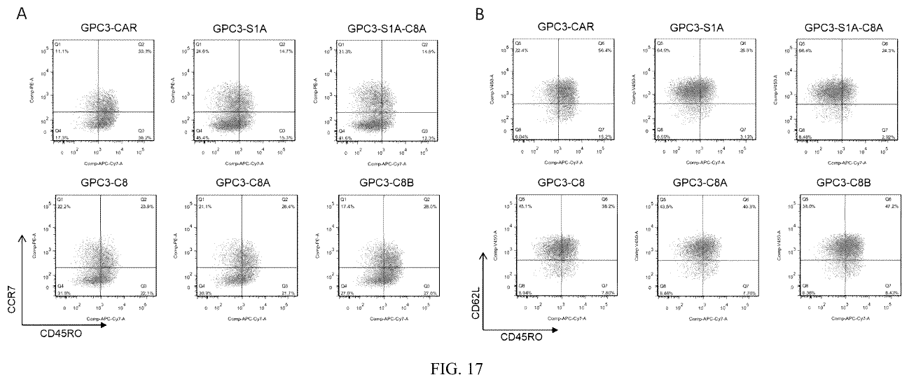

The T cells expressing GPC3-CAR, GPC3-S1A, GPC3-C8, GPC3-C8A, GPC3-C8B or GPC3-S1A-C8A obtained in Example 2 were cultured in vitro in a 37° C., 5% CO₂ cell culture incubator for 9 or 12 days. The expression of CD3, CD45RO, CD45RA, CD62L, CCR7, CD95, CD122, CD127, CD27 and CD28 proteins in T cells was detected by BD flow cytometer. As shown in FIG. 17, the proportion of memory stem cell-like T cells (TSCM) in the CD3 cells expressing GPC3-S1A, GPC3-C8, GPC3-C8A, GPC3-C8B or GPC3-S1A-C8A was significantly higher than that in the GPC3-CAR control group.

### Example 16: The Combination of CD258 Protein and SIRT1 Protein Promotes the Proliferation of Memory T Cells in CD4 and CD8 Cells (GPC3)

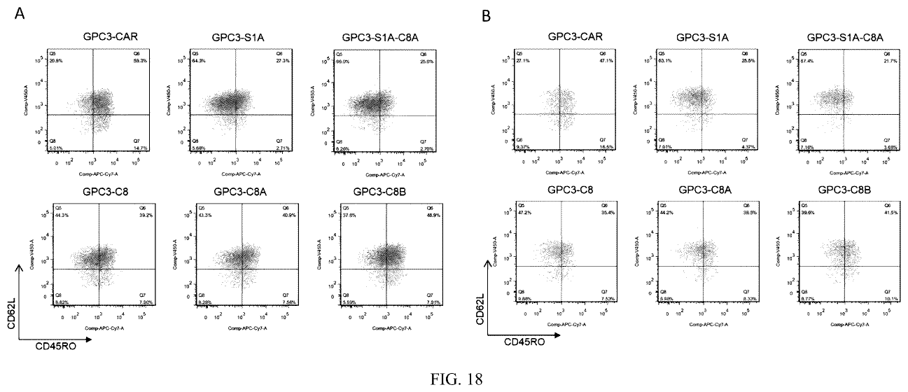

T cells expressing GPC3-CAR, GPC3-S1A, GPC3-C8, GPC3-C8A, GPC3-C8B or GPC3-S1A-C8A obtained in Example 2 were cultured in vitro in a 37° C., 5% CO₂ cell culture incubator for 9 or 12 days. The expression of CD4, CD45RO, CD45RA, CD62L, CCR7, CD95, CD122, CD127, CD27, and CD28 proteins in T cells was detected by BD flow cytometer. As shown in FIG. 18A, the proportion of memory stem cell-like T cells (TSCM) in the CD4 cells expressing GPC3-S1A, GPC3-C8, GPC3-C8A, GPC3-C8B, or GPC3-S1A-C8A was significantly higher than that in the GPC3-CAR control group. Similarly, the T cells expressing GPC3-CAR, GPC3-S1A, GPC3-C8, GPC3-C8A, GPC3-C8B or GPC3-S1A-C8A obtained in Example 2 were cultured in vitro at 37° C. and 5% CO₂ in a cell culture incubator for 9 or 12 days. The expression of CD8, CD45RO, CD45RA, CD62L, CCR7, CD95, CD122, CD127, CD27 and CD28 proteins in T cells was detected by BD flow cytometer. As shown in FIG. 18B, the proportion of memory stem cell-like T cells (TSCM) in the CD8 cells expressing GPC3-S1A, GPC3-C8, GPC3-C8A, GPC3-C8B or GPC3-S1A-C8A was significantly higher than that in the GPC3-CAR control group.

### Example 17: The Combination of CD258 Protein and SIRT1 Protein Inhibits the Expression of Negative Immune Regulatory Proteins (CD19)

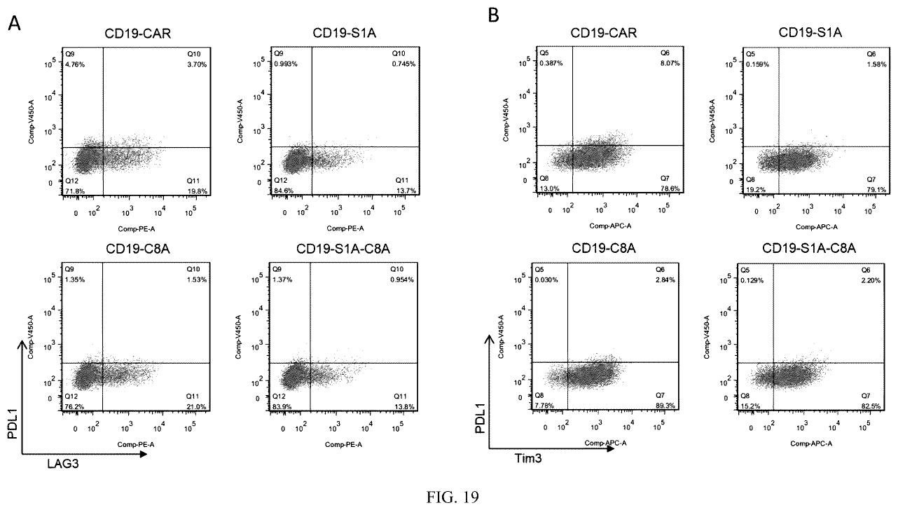

The T cells expressing CD19-CAR, CD19-S1A, CD19-C8A or CD19-S1A-C8A obtained in Example 2 were cultured in vitro in a cell culture incubator at 37° C. and 5% CO₂ for 9 or 12 days. The expression of CD3, CD4, CD8, PD1, PDL1, TIM3 and LAG3 proteins in T cells was detected by BD flow cytometer. The results are shown in FIG. 19. In T cells expressing CD19-S1A, CD19-C8A or CD19-S1A-C8A, the proportion of cells expressing two negative regulatory proteins at the same time was significantly lower than that in the CD19-CAR control group.

### Example 18: The Combination of CD258 Protein and SIRT1 Protein Promotes the Proliferation of Memory T Cells in CD8 Cells (CD19)

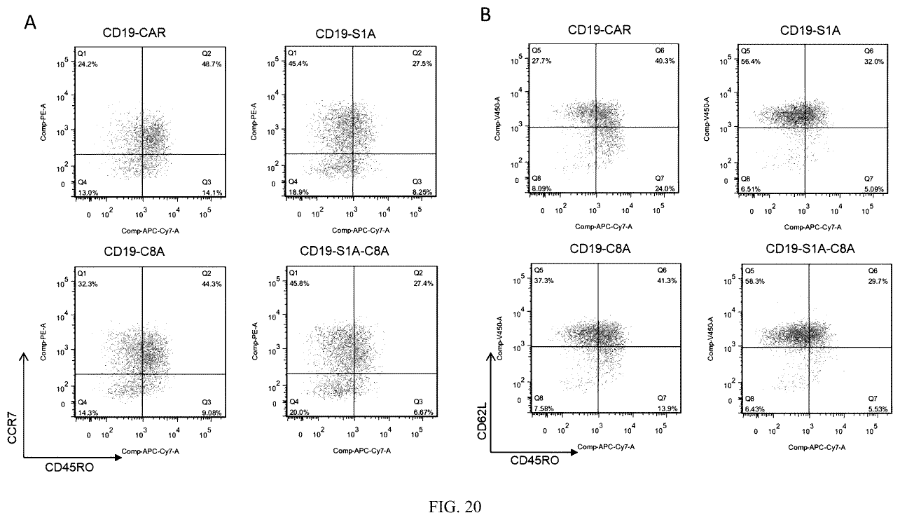

The T cells expressing CD19-CAR, CD19-S1A, CD19-C8A, or CD19-S1A-C8A obtained in Example 2 were cultured in vitro in a 37° C., 5% CO₂ cell culture incubator for 9 or 12 days. The expression of CD8, CD45RO, CD45RA, CD62L, CCR7, CD95, CD122, CD127, CD27 and CD28 proteins in T cells was detected by BD flow cytometer. As shown in FIG. 20, the proportion of memory stem cell-like T cells (TSCM) in the total cells in CD8 cells expressing CD19-S1A, CD19-C8A or CD19-S1A-C8A was significantly higher than that in the CD19-CAR control group.

### Example 19: The Combination of CD258 Protein and SIRT1 Protein Promotes the Proliferation of Memory T Cells in CD3 and CD4 Cells (CD19)

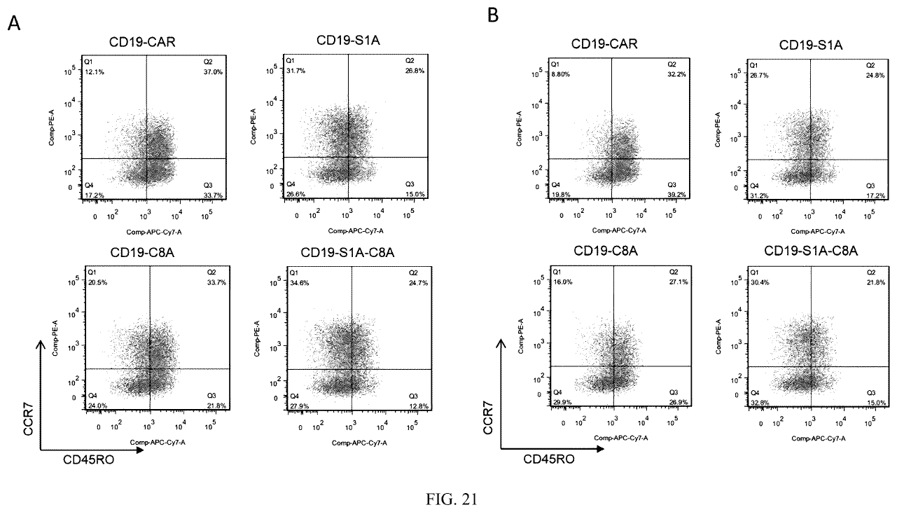

The T cells expressing CD19-CAR, CD19-S1A, CD19-C8A or CD19-S1A-C8A respectively obtained in Example 2 were cultured in vitro in a 37° C., 5% CO₂ cell culture incubator for 9 or 12 days. The expression of CD3, CD45RO, CD45RA, CD62L, CCR7, CD95, CD122, CD127, CD27, and CD28 proteins in T cells was detected by BD flow cytometer. As shown in FIG. 21A, the proportion of memory stem cell-like T cells (TSCM) in the total cells in CD3 cells expressing CD19-S1A, CD19-C8A or CD19-S1A-C8A was significantly higher than that in the CD19-CAR control group. Similarly, the T cells expressing CD19-CAR, CD19-S1A, CD19-C8A or CD19-S1A-C8A obtained in Example 2 were cultured in vitro in a 37° C., 5% CO₂ cell culture incubator for 9 or 12 days. The expression of CD4, CD45RO, CD45RA, CD62L, CCR7, CD95, CD122, CD127, CD27, and CD28 proteins in T cells was detected by BD flow cytometer. As shown in FIG. 21B, the proportion of memory stem cell-like T cells (TSCM) in the total cells in CD4 cells expressing CD19-S1A, CD19-C8A or CD19-S1A-C8A was also significantly higher than that in the CD19-CAR control group.

### Example 20: The Combination of CD258 Protein and SIRT1 Protein Inhibits the Expression of Immune Negative Regulatory Proteins (MSLN)

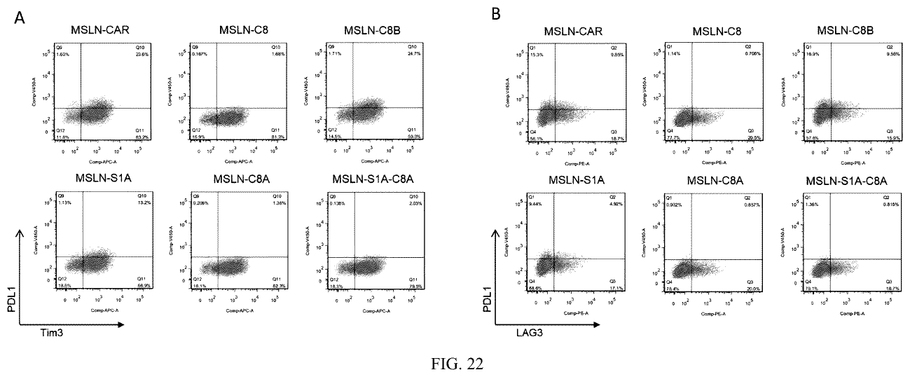

The T cells expressing MSLN-CAR, MSLN-C8, MSLN-C8A, MSLN-C8B, MSLN-S1A or MSLN-S1A-C8A respectively obtained in Example 2 were cultured in vitro in a 37° C., 5% CO₂ cell culture incubator for 9 or 12 days. The expression of CD3, CD4, CD8, PD1, PDL1, TIM3, and LAG3 proteins in T cells was detected by BD flow cytometer. The results are shown in FIG. 22. In T cells expressing MSLN-C8, MSLN-C8A, MSLN-C8B, MSLN-S1A or MSLN-S1A-C8A, the proportion of cells expressing two negative regulatory proteins at the same time was significantly lower than that of the MSLN-CAR control group.

### Example 21: The Combination of CD258 Protein and SIRT1 Protein Promotes the Proliferation of Memory T Cells in CD3 Cells (MSLN)

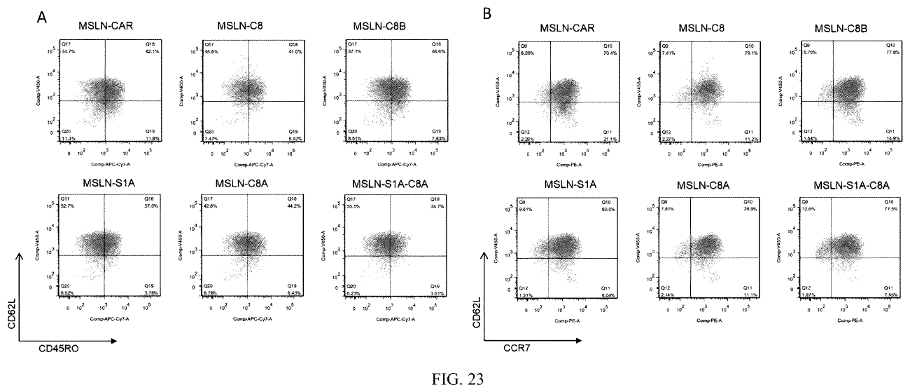

The T cells expressing MSLN-CAR, MSLN-C8, MSLN-C8A, MSLN-C8B, MSLN-S1A or MSLN-S1A-C8A respectively obtained in Example 2 were cultured in vitro in a 37° C., 5% CO₂ cell culture incubator for 9 or 12 days. The expression of CD3, CD45RO, CD45RA, CD62L, CCR7, CD95, CD122, CD127, CD27 and CD28 proteins in T cells was detected by BD flow cytometer. As shown in FIG. 23, the proportion of memory stem cell-like T cells (TSCM) in the CD3 cells expressing MSLN-C8, MSLN-C8A, MSLN-C8B, MSLN-S1A or MSLN-S1A-C8A was significantly higher than that in the MSLN-CAR control group.

### Example 22: The Combination of CD258 Protein and SIRT1 Protein Enhances the Killing Ability of CAR-T Cells Against Tumor Cells

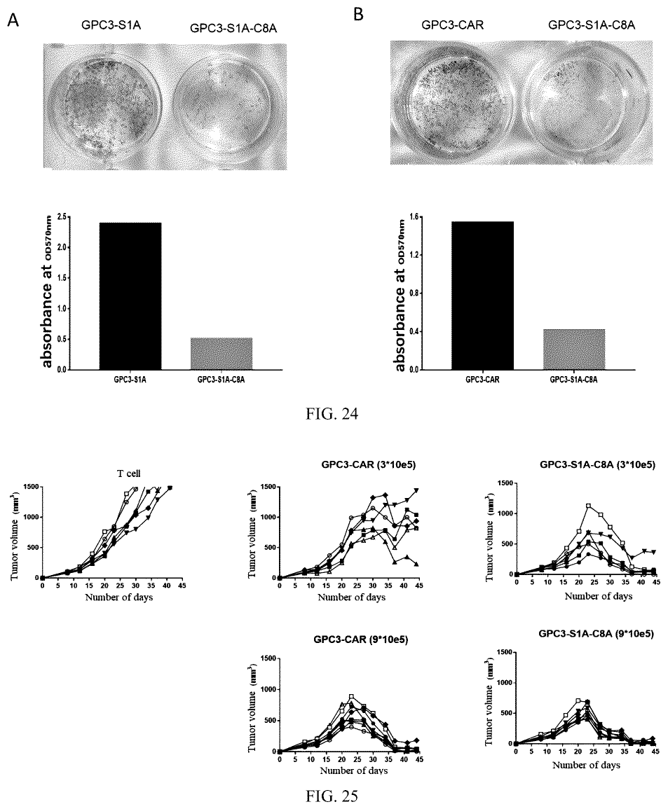

T cells expressing GPC3-S1A or GPC3-S1A-C8A were co-cultured with Huh7 cells (purchased from the cell bank of the Chinese Academy of Sciences) at a cell number ratio of 1:15 using Xvivo15 medium for 7 days, and crystal violet staining (MCE) was used to detect the killing effect of CAR-T cells on tumor cells. The results are shown in FIG. 24A. The killing effect of T cells expressing GPC3-S1A-C8A on tumor cells was much higher than that of T cells expressing GPC3-S1A in the control group.

Similarly, T cells expressing GPC3-CAR or GPC3-S1A-C8A were co-cultured with Huh7 cells (purchased from the cell bank of the Chinese Academy of Sciences) at a cell number ratio of 1:15 for 7 days using Xvivo15 medium, and crystal violet staining (MCE) was used to detect the killing effect of CAR-T cells on tumor cells. The results are shown in FIG. 24B. The killing effect of T cells expressing GPC3-S1A-C8A on tumor cells is also much higher than that of T cells expressing GPC3-CAR in the control group.

### Example 23: The Combination of CD258 Protein and SIRT1 Protein Enhances the Anti-Tumor Effect of CAR-T Cells

Huh7 cells (1×10⁷/mouse) were subcutaneously inoculated into NSG mice (purchased from Biocytogen). After 14 days, the tumor volume of the mice was measured to be about 200 mm³. At this time, the mice were divided into 5 groups, namely T, GPC3-CAR and GPC3-S1A-C8A groups, with 6-8 mice in each group. Then, T cells (9×10⁵/mouse) were injected into the T cell group through the tail vein, CAR-T cells expressing GPC3-CAR were injected into the GPC3-CAR group (3×10⁵/mouse or 9×10⁵/mouse), and CAR-T cells expressing GPC3-S1A-C8A were injected into the GPC3-S1A-C8A group (3×10⁵/mouse or 9×10⁵/mouse). The tumor volume was measured on Mondays and Thursdays of each week, and the death of mice was recorded. As shown in FIG. 25, the tumor inhibition effect of the GPC3-S1A-C8A group was significantly higher than that of the other control groups.

### Example 24: The Combination of CD258 Protein and SIRT1 Protein Promotes the Proliferation of Granulocytes and Monocytes In Vivo

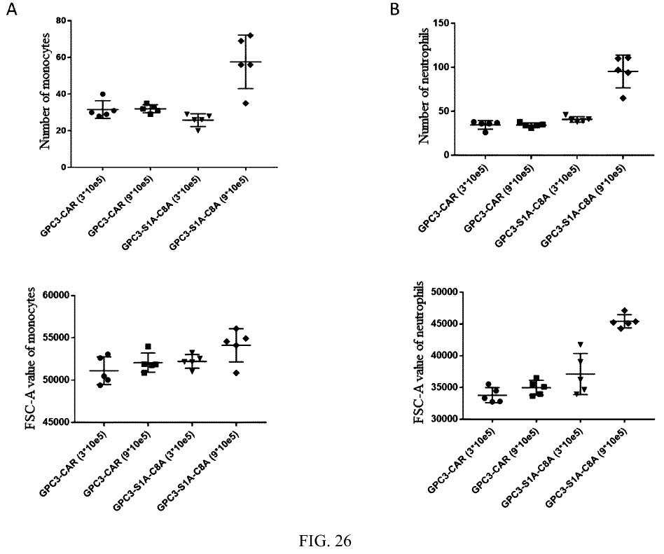

Huh7 cells (1×10⁷/mouse) were subcutaneously inoculated into NSG mice (purchased from Biocytogen). After 14 days, the tumor volume of the mice was measured to be about 200 mm³. At this time, the mice were divided into 5 groups, namely T, GPC3-CAR and GPC3-S1A-C8A groups, with 6 mice in each group. Then, T cells (9×10⁵/mouse) were injected into the T cell group through the tail vein, CAR-T cells expressing GPC3-CAR (3×10⁵/mouse or 9×10⁵/mouse) were injected into the GPC3-CAR group, and CAR-T cells expressing GPC3-S1A-C8A (3×10⁵/mouse or 9×10⁵/mouse) were injected into the GPC3-S1A-C8A group. On the 7th day, 50 μl of blood was collected from the tail of the mouse, and the number and size of monocytes and neutrophils in each group were detected by BD flow cytometry. As shown in FIG. 26, the number and size of monocytes and neutrophils in the GPC3-S1A-C8A group were much higher than those in the GPC3-CAR control group.

### Example 25: The Combination of CD258 Protein and SIRT1 Protein Promotes the Proliferation of CAR-T Cells In Vivo and the Release of Th1 Cytokines

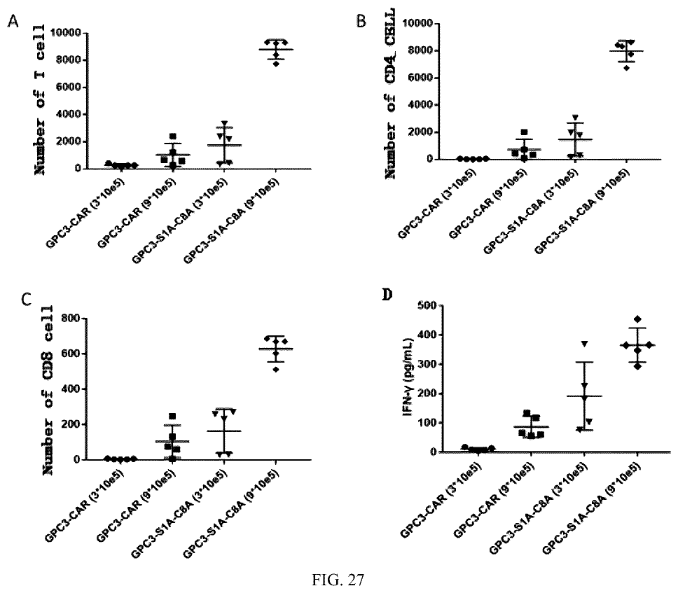

Huh7 cells (1×10⁷/mouse) were subcutaneously inoculated into NSG mice (purchased from Biocytogen). After 14 days, the tumor volume of the mice was measured to be about 200 mm³. At this time, the mice were divided into 5 groups, namely T, GPC3-CAR and GPC3-S1A-C8A groups, with 6 mice in each group. Then, T cells (9×10⁵/mouse) were injected into the T cell group through the tail vein, CAR-T cells expressing GPC3-CAR were injected into the GPC3-CAR group (3×10⁵/mouse or 9×10⁵/mouse), and CAR-T cells expressing GPC3-S1A-C8A were injected into the GPC3-S1A-C8A group (3×10⁵/mouse or 9×10⁵/mouse). On the 14th day, 50 μl of blood was collected from the tail of the mice, and the expression of human CD3, CD4 and CD8 proteins in each group was detected by BD flow cytometry. The results are shown in FIGS. 27A, 27B and 27C. In the groups expressing CD3, CD4 and CD8, the proportion of cells expressing CD3, CD4 or CD8 proteins in the GPC3-S1A-C8A group to the total number of cells was much higher than that in the GPC3-CAR control group.

Similarly, Huh7 cells (1×10⁷/mouse) were subcutaneously inoculated into NSG mice (purchased from Biocytogen). After 14 days, the tumor volume of the mice was measured to be about 200 mm³. At this time, the mice were divided into 5 groups, namely T, GPC3-CAR and GPC3-S1A-C8A groups, with 6 mice in each group. Then, T cells (9×10⁵/mouse) were injected into the T cell group through the tail vein, CAR-T cells expressing GPC3-CAR (3×10⁵/mouse or 9×10⁵/mouse) were injected into the GPC3-CAR group, and CAR-T cells expressing GPC3-S1A-C8A (3×10⁵/mouse or 9×10⁵/mouse) were injected into the GPC3-S1A-C8A group. On the 7th day, 50 μl of blood was collected from the tail of the mice, and the expression of human IL-2, IL4, IL6, IL10, TNF-α and IFN-γ cytokines in each group was detected by BD flow cytometry. The results are shown in FIG. 27D. The expression of IFN-γ cytokine in the GPC3-S1A-C8A group was significantly higher than that in the GPC3-CAR control group.

What is claimed is:

1. A n in vivo method or an in vitro method selected from the group consisting of:
(1) a method to promote the proliferation of immune cell, (2) a method to promote the production of memory immune cells, (3) a method to inhibit the differentiation of immune cell, (4) a method to inhibit the expression of immune negative regulatory proteins in immune cells, (5) a method for enhancing immune cells to release cytokines, and (6) a method for enhancing the ability of immune cells to kill tumors,
the method includes the following steps: up-regulating the expression level of one or the combination of more of SIRT1 or the functional mutant thereof, SIRT2 or the functional mutant thereof, SIRT3 or the functional mutant thereof, SIRT4 or the functional mutant thereof, SIRT5 or the functional mutant thereof, SIRT6 or the functional mutant thereof, SIRT7 protein or the functional mutant thereof in the immune cells,
or up-regulation of the expression level of the CD258 protein or the functional mutant thereof and one or the combination of SIRT1 or the functional mutant thereof, SIRT2 or the functional mutant thereof, SIRT3 or functional mutants thereof, SIRT4 or functional mutants thereof, SIRT5 or functional mutants thereof, SIRT6 or functional mutants thereof, SIRT7 protein or functional mutants thereof in the immune cells;
or a method selected from the group consisting of:
(a) a method to solve tumor heterogeneity in subjects, (b) a method to prevent recurrence of tumors in subjects, and (c) a method to treat tumors in subjects in need,
the method comprises administering immune cells to the subject, wherein up-regulating the expression level of one or the combination of more of SIRT1 or the functional mutant thereof, SIRT2 or the functional mutant thereof, SIRT3 or the functional mutant thereof, SIRT4 or the functional mutant thereof, SIRT5 or the functional mutant thereof, SIRT6 or the functional mutant thereof, SIRT7 protein or the functional mutant thereof in the immune cells,
or up-regulation of the expression level of the CD258 protein or the functional mutant thereof and one or the combination of SIRT1 or the functional mutant thereof, SIRT2 or the functional mutant thereof, SIRT3 or functional mutants thereof, SIRT4 or functional mutants thereof, SIRT5 or functional mutants thereof, SIRT6 or functional mutants thereof, SIRT7 protein or functional mutants thereof in the immune cells.

2. The method of claim 1, wherein the tumor is selected from liver cancer, lung cancer, leukemia and mesothelioma, the negative immune regulatory protein is selected from PD1, PDL1, TIM3 and LAG3, and the cytokine is selected from interleukin, interferon and/or tumor necrosis factor, preferably, the cytokine is selected from IL-2, IL4, IL6, IL7, IL10, IL12, TNF-α and/or IFNγ; the immune cell is a lymphocyte; preferably, the immune cells are T cells, B cells, natural killer cells, immature dendritic cells, monocytes and macrophages; further preferably, the T cells are selected from memory stem cell-like T cells and/or central memory T cells; preferably, the TSCM is CCR7⁺ and/or CD62L⁺, preferably, the TSCM also has one or more properties selected from: CD45RA⁺ or CD45RA⁻, CD45RO⁺ or CD45RO⁻, CD27⁺, CD28⁺, CD127⁺, CD122⁺, CD95⁺, CD3⁺, CD4⁺ and CD8⁺.

3. The method of claim 1, wherein the immune cells are selected from genetically modified immune cells, and the genetically modified immune cells express chimeric antigen receptors or T cell receptors, preferably the genetically modified immune cells are genetically modified T cells; preferably, the method includes the steps of isolating and activating the genetically modified immune cells, wherein the activation includes administering T cell culture medium to the isolated genetically modified immune cells, preferably, the T cell culture medium is selected from one or more of the following: DMEM culture medium, 1640 culture medium, MEM culture medium, X-VIVO culture medium and stem cells culture medium.

4. The method of claim 1, wherein the TCR comprises a subunit selected from: TCRα, TCRβ, TCRγ and TCRδ,
preferably, the subunits of the TCR include a variable region of extracellular domain that specifically binds and/or recognizes tumor antigens, preferably, the variable region of extracellular domain is selected from: variable region fragment Vα of TCRα, variable region fragment Jα of TCRα, variable region fragment Vβ of TCRβ, variable region fragment Dβ of TCRβ and variable region fragment Jβ of TCRβ,
preferably, the extracellular domain of the variable region specifically binds and/or recognizes a target selected from MAGEA family members, CTA family members, HPV viruses and tyrosinase, preferably, the extracellular domain of the variable region specifically binds and/or recognizes a target selected from: MAGEA3, MAGEA4, NY-ESO-1, MART1, HPV16-E6 and melanoma antigen tyrosinase.

5. The method of claim 1, the CAR comprising an intracellular domain which includes a signaling domain and/or a costimulatory domain,
preferably, the signaling domain is selected from: the signaling domain of CD3ζ having the nucleotide sequence shown in SEQ ID NO: 11 and the amino acid sequence shown in SEQ ID NO: 83, the signaling domain of CD38 and the signaling domain of CD3ε; preferably, the signaling domain comprises or consists of the sequence shown as SEQ ID NO: 83 or is at least 80%, at least 85%, at least 90%, at least 91%, at least 92%, at least 93%, at least 94%, at least 95%, at least 96%, at least 97%, at least 98% or at least 99% homology thereto, and the homologous sequence still has the function of the sequence shown as SEQ ID NO: 83,
preferably, the costimulatory domain refers to a functional signaling domain of a protein selected from one or more of the following: CD27, CD28, 4-1BB (CD137), OX40, CD30, CD40, PD-1, ICOS, lymphoid Cell function-associated antigen 1 (LFA-1), CD2, CD7, LIGHT, NKG2C, B7-H3, ligand that specifically binds to CD83, CDS, ICAM-1, GITR, BAFFR, HVEM (LIGHTR), SLAMF7, NKp80 (KLRF1), CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46 and NKG2D; preferably, the costimulatory domain is selected from: CD27, CD28 and 4-1BB;
more preferably, the costimulatory domain is selected from: the costimulatory domain of CD27, the costimulatory domain of CD28 having the nucleotide sequence is shown in as SEQ ID NO: 9 and the amino acid sequence shown in SEQ ID NO: 81 and the costimulatory domain of 4-1BB having the nucleotide sequence is shown as SEQ ID NO: 10 and the amino acid sequence shown in SEQ ID NO: 82; preferably, the costimulatory domain contains or consists of the sequence shown in any of: SEQ ID NO: 81 and SEQ ID NO: 82, or is at least 80%, at least 85%, at least 90%, at least 91%, at least 92%, at least 93%, at least 94%, at least 95%, at least 96%, at least 97%, at least 98% or at least 99% homology thereto, and the homologous sequence still has the function of the sequence shown as SEQ ID NO: 81 or SEQ ID NO: 82,
preferably, the CAR comprises a hinge region, and more preferably the hinge region is selected from: the hinge region of IgG4, the hinge region of IgG1 and the hinge region of CD8 having the nucleotide sequence shown in SEQ ID NO:7 and the amino acid sequence shown in SEQ ID NO: 79; preferably, the hinge region includes or consists of the sequence shown in SEQ ID NO: 79,
preferably, the CAR comprises a transmembrane region, preferably, the transmembrane region is selected from: the transmembrane region of CD8 having the nucleotide sequence is shown as SEQ ID NO: 8 and the amino acid sequence shown in SEQ ID NO: 80, the transmembrane region of CD28 and the transmembrane region of CD24, preferably, the transmembrane region includes or consists of the sequence shown in SEQ ID NO: 80,
preferably, the CAR includes a targeting moiety, preferably the targeting moiety specifically binds and/or recognizes a tumor antigen,
preferably, the tumor antigen include TSHR, CD19, CD123, CD138, CD22, CD30, CD171, CS-1, CLL-1, CD33, EGFRVIII, GD2, GD3, BCMA, TnAg, PSMA, ROR1, FLT3, FAP, TAG72, CD38, CD44v6, CEA, EPCAM, B7H3, KIT, IL-13Ra2, mesothelin, IL-11Ra, PSCA, PRSS21, VEGFR2, LewisY, CD24, PDGFR-β, SSEA-4, CD20, folate receptor α, ERBB2 (Her2/neu), MUC1, EGFR, NCAM, Prostase, PAP, ELF2M, ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, folate receptor beta, TEM1/CD248, TEM7R, CLDN6, GPRC5D, CXORF61, CD97, CD179a, ALK, polysialic acid, PLACI, GloboH, NY-BR-1, UPK2, HAVCR1, ADRB3, PANX3, GPR20, LY6K, OR51E2, TARP, WT1, NY-ESO-1, LAGE-la, MAGE-A1, legumain, HPVE6, E7, MAGE A1, ETV6-AML, sperm protein 17, XAGE1, ie 2, MAD-CT-1, MAD-CT-2, Fos-related antigen 1, p53, p53 mutants, prostein, survivin and telomerase, PCTA-1/Galectin8, MelanA/MART1, Ras mutant, hTERT, sarcoma translocation breakpoint, ML-IAP, ERG (TMPRSS2ETS fusion gene), NA17, PAX3, androgen receptor, cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxylesterase, mut hsp702, CD79a, CD79b, CD72, LAIR1, FCAR, LILRA2, CD300LF, CLEC12A, BST2, EMR2, LY75, GPC3, FCRL5 and IGLL1,
more preferably, the targeting moiety specifically binds and/or recognizes a target selected from: B lymphocyte surface antigens, TNF family members, HER family members, and GPC family members, and further preferably, the targeting moiety specifically binds and/or recognizes a target selected from: CD19, BCMA, HER2, Mesothelin and GPC3.

6. The method of claim 5, wherein the targeting moiety is scFv, preferably the targeting moiety is selected from scFv against GPC3, scFv against CD19, scFv against BCMA, scFv against MSLN, scFv against HER2 scFv,
preferably, the scFv against GPC3 includes LCDR1 (preferably according to the Kabat numbering system, the sequence shown in SEQ ID NO: 36), LCDR2 (preferably according to the Kabat numbering system, the sequence shown in SEQ ID NO: 37) and LCDR3 (preferably according to the Kabat numbering system, the sequence as shown in SEQ ID NO: 38) contained in the light chain variable region shown in SEQ ID NO: 35 and HCDR1 (preferably according to the Kabat numbering system, the sequence shown in SEQ ID NO: 40), HCDR2 (preferably according to the Kabat numbering system, the sequence shown in SEQ ID NO: 41) and HCDR3 (preferably according to the Kabat numbering system, the sequence shown in SEQ ID NO: 42) contained in the heavy chain variable region shown in SEQ ID NO: 39, more preferably, the scFv against GPC3 comprises or consists of the nucleotide sequence shown in SEQ ID NO: 2 or the amino acid sequence shown in SEQ ID NO: 34, or has at least 80%, at least 85%, at least 90%, at least 91%, at least 92%, at least 93%, at least 94%, at least 95%, at least 96%, at least 97%, at least 98% or at least 99% homology thereto, and the homologous sequence still has the function of the sequence shown as SEQ ID NO: 2 or SEQ ID NO: 34;
the scFv against CD19 includes LCDR1 (preferably according to the Kabat numbering system, the sequence shown in SEQ ID NO: 45), LCDR2 (preferably according to the Kabat numbering system, the sequence shown in SEQ ID NO: 46) and LCDR3 (preferably according to the Kabat numbering system, the sequence shown in SEQ ID NO: 47) contained in the light chain variable region shown in SEQ ID NO: 44 and HCDR1 (preferably according to the Kabat numbering system, the sequence shown in SEQ ID NO: 49), HCDR2 (preferably according to the Kabat numbering system, the sequence shown in SEQ ID NO: 50) and HCDR3 (preferably according to the Kabat numbering system, the sequence shown in SEQ ID NO: 51) contained in the heavy chain variable region shown in SEQ ID NO: 48, more preferably, the scFv against CD19 comprises or consists of the nucleotide sequence as shown in SEQ ID NO: 3 or the amino acid sequence as shown in SEQ ID NO: 43 or has at least 80%, at least 85%, at least 90%, at least 91%, at least 92%, at least 93%, at least 94%, at least 95%, at least 96%, at least 97%, at least 98% or at least 99% homology thereto, the sequence still has the function of the sequence shown in SEQ ID NO: 3 or SEQ ID NO: 43;
the scFv against BCMA includes LCDR1 (preferably according to the Kabat numbering system, the sequence shown in SEQ ID NO: 54), LCDR2 (preferably according to the Kabat numbering system, the sequence shown in SEQ ID NO:55) and LCDR3 (preferably according to the Kabat numbering system, the sequence shown in SEQ ID NO:56) contained in the light chain variable region shown in SEQ ID NO: 53 and HCDR1 (preferably according to the Kabat numbering system, the sequence shown in SEQ ID NO: 58), HCDR2 (preferably according to the Kabat numbering system, the sequence shown in SEQ ID NO: 59) and HCDR3 (preferably according to the Kabat numbering system, the sequence shown in SEQ ID NO: 60) contained in the heavy chain variable region shown in SEQ ID NO: 57, more preferably, the scFv against BCMA contains or consists of the nucleotide sequence as shown in SEQ ID NO:4 or the amino acid sequence shown in SEQ ID NO:52 or has at least 80%, at least 85%, at least 90%, at least 91%, at least 92%, at least 93%, at least 94%, at least 95%, at least 96%, at least 97%, at least 98% or at least 99% homology thereto, the sequence still has the function of the sequence shown in SEQ ID NO: 4 or SEQ ID NO: 52;
the scFv against MSLN includes LCDR1 (preferably according to the Kabat numbering system, the sequence shown in SEQ ID NO: 63), LCDR2 (preferably according to the Kabat numbering system, the sequence shown in SEQ ID NO: 64) and LCDR3 (preferably according to the Kabat numbering system, the sequence shown in SEQ ID NO:65) contained in the light chain variable region shown in SEQ ID NO: 62 and HCDR1 (preferably according to the Kabat numbering system, the sequence shown in SEQ ID NO: 67), HCDR2 (preferably according to the Kabat numbering system, the sequence shown in SEQ ID NO: 68) and HCDR3 (preferably according to the Kabat numbering system, the sequence shown in SEQ ID NO: 69) comprising the heavy chain variable region shown in SEQ ID NO: 66, more preferably, the scFv against MSLN comprises or consists of the nucleotide sequence as shown in SEQ ID NO: 5 or the amino acid sequence shown in SEQ ID NO: 61 or has at least 80%, at least 85%, at least 90%, at least 91%, at least 92%, at least 93%, at least 94%, at least 95%, at least 96%, at least 97%, at least 98% or at least 99% homology thereto, the sequence still has the function of the sequence shown as SEQ ID NO: 5 or SEQ ID NO: 61;
the scFv against HER2 includes LCDR1 (preferably according to the Kabat numbering system, the sequence is shown as SEQ ID NO: 72), LCDR2 (preferably according to the Kabat numbering system, the sequence shown in SEQ ID NO: 73) and LCDR3 (preferably according to the Kabat numbering system, the sequence shown in SEQ ID NO: 74) contained in the light chain variable region shown in SEQ ID NO: 71 and HCDR1 (preferably according to the Kabat numbering system, the sequence shown in SEQ ID NO: 76), HCDR2 (preferably according to the Kabat numbering system, the sequence shown in SEQ ID NO: 77) and HCDR3 (preferably according to the Kabat numbering system, the sequence shown in SEQ ID NO: 78) contained in the heavy chain variable region shown in SEQ ID NO: 75, more preferably, the scFv against HER2 comprises or consists of the nucleotide sequence as shown in SEQ ID NO: 6 or the amino acid sequence shown in SEQ ID NO: 70 or has at least 80%, at least 85%, at least 90%, at least 91%, at least 92%, at least 93%, at least 94%, at least 95%, at least 96%, at least 97%, at least 98% or at least 99% homology thereto, the sequence still has the function of the sequence shown as SEQ ID NO: 6 or SEQ ID NO: 70.

7. The method of claim 1, wherein the method further Includes-includes the following steps:
isolate and obtain peripheral blood mononuclear cells PBMC, CD3⁺ T lymphocytes, CD8⁺ T lymphocytes, CD4⁺ T lymphocytes or regulatory T cells, preferably, the method further includes: adding one or more T cell stimulating factors to the isolated PBMCs, preferably, the T cell stimulating factors are selected from: antibodies against the surface antigens of B lymphocyte, anti-TNF antibodies, intracellular polyesters and antibiotics, preferably, the T cell stimulating factor is selected from: anti-CD3 antibodies, anti-CD28 antibodies, anti-4-1BB antibody, anti-CD80 antibody, anti-CD86 antibody, PHA, PMA and ionomycin, more preferably, the T cell stimulating factor is an anti-CD3 antibody, and the concentration of the anti-CD3 antibody is 1-10000 ng/mL, or the T cell stimulating factor is an anti-CD28 antibody, and the concentration of the anti-CD28 antibody is 1-10000 ng/mL.

8. The method of claim 1, wherein the method further includes:
adding one or more cytokines to the isolated PBMC, preferably, the cytokine is interleukin, more preferably, the interleukin is IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35 and/or IL-36; more preferably, the interleukin is IL2, and the concentration of IL2 is 0.1-10000 U/mL, or the interleukin is IL21, and the concentration of IL21 is 0.01-1000 ng/mL, or the interleukin is IL7, and the concentration of IL7 is 0.01-1000 ng/mL, or the interleukin is IL15, and the concentration of IL15 is 0.01-1000 ng/mL.

9. The method of claim 1, wherein the SIRT1 or the functional mutant thereof, SIRT2 or the functional mutant thereof, SIRT3 or the functional mutant thereof, SIRT4 or the functional mutant thereof, SIRT5 or the functional mutant thereof, SIRT6 or the functional mutant thereof, SIRT7 protein or the functional mutant thereof, CD258 or the functional mutant thereof are derived from a human.

10. The method of claim 1, wherein the SIRT1 protein, SIRT2 protein, SIRT3 protein, SIRT4 protein, SIRT5 protein, SIRT6 protein, and SIRT7 protein respectively comprise or consisted of the sequence shown in any one of the following: SEQ ID NO: 15, SEQ ID NO: 16, SEQ ID NO: 17, SEQ ID NO: 18, SEQ ID NO: 19, SEQ ID NO: 20, SEQ ID NO: 21 or SEQ ID NO: 86, SEQ ID NO: 87, SEQ ID NO: 88, SEQ ID NO: 89, SEQ ID NO: 90, SEQ ID NO: 91, SEQ ID NO: 92, or at least 80%, at least 85%, at least 90%, at least 91%, at least 92%, at least 93%, at least 94%, at least 95%, at least 96%, at least 97%, at least 98%, or at least 99% homology thereto, the sequence still has the function of the sequence shown in SEQ ID NO: 15, SEQ ID NO: 16, SEQ ID NO: 17, SEQ ID NO: 18, SEQ ID NO: 19, SEQ ID NO: 20, SEQ ID NO: 21 or SEQ ID NO: 86, SEQ ID NO: 87, SEQ ID NO: 88, SEQ ID NO: 89, SEQ ID NO: 90, SEQ ID NO: 91, SEQ ID NO: 92,
the functional mutant of the SIRT1 protein is selected from:
(1) the functional mutants of the SIRT1 protein, SIRT2 protein, SIRT3 protein, SIRT4 protein, SIRT5 protein, SIRT6 protein, and SIRT7 protein have mutations in the domains of the SIRT1 protein, SIRT2 protein, SIRT3 protein, SIRT4 protein, SIRT5 protein, SIRT6 protein, and SIRT7 protein selected from: sirtuin domain, the small molecule Sirtuin activator binding domain, and/or the C-terminal regulatory domain,
(2) the SIRT1 protein, wherein any one or more amino acids in the sequence shown in SEQ ID NO: 105 and the sequence shown in SEQ ID NO: 106 are substituted by A to obtain the functional mutant, and the substitution results in the loss of function of the protein having the sequence shown in SEQ ID NO: 105 and the loss of function of the protein having the sequence shown in SEQ ID NO: 106, and the more preferred functional mutants are PLRKRPAA as shown in SEQ ID NO: 107 and PPKRAAAA as shown in SEQ ID NO: 108,
(3) the SIRT1 protein, wherein functional mutants are obtained by completely deleting the sequence PLRKRPRR as shown in SEQ ID NO: 105 and the sequence PPKRKKRK as shown in SEQ ID NO: 106,
(4) the SIRT1 protein, wherein any one or more amino acids in the sequence shown in SEQ ID NO: 109 and the sequence shown in SEQ ID NO: 110 are replaced with A to obtain the functional mutant, and the substitution results in the loss of function of the protein having that the sequence shown in SEQ ID NO: 109 and the loss of function of the protein having the sequence shown in SEQ ID NO: 110, and the more preferred mutants are AAATDGAA as shown in SEQ ID NO: 111 and ADAAAA as shown in SEQ ID NO: 112,
(5) the SIRT1 protein, wherein a functional mutant is obtained by completely deleting the sequence shown in SEQ ID NO: 109 and the sequence shown in SEQ ID NO: 110,
more preferably, the SIRT1 functional mutant comprises or consists of the sequence shown in any one of the following: SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30 or SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, or is at least 80%, at least 85%, at least 90%, at least 91%, at least 92%, at least 93%, at least 94%, at least 95%, at least 96%, at least 97%, at least 98%, or at least 99% homologous thereto, the homologous sequence still has the function of the sequence shown in any one of SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30 or SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101.

11. The method of claim 1, wherein the CD258 protein comprises or consists of the sequence shown as SEQ ID NO: 31 or SEQ ID NO: 102 or is at least 80%, at least 85%, at least 90%, at least 91%, at least 92%, at least 93%, at least 94%, at least 95%, at least 96%, at least 97%, at least 98%, or at least 99% homologous thereto, and the homologous sequences still has the function of the sequence shown in SEQ ID NO: 31 or SEQ ID NO: 102,
the CD258 protein functional mutant is selected from:
(1) a functional mutant obtained by replacing one or both of Q and L at positions 82-83 with A (preferably the QL amino acids are replaced with AA) in the sequence shown as SEQ ID NO: 102, and the functional mutation makes the protein always expressed on the cell membrane in a membrane-bound form and reduces the expression of the secreted form of the protein;
(2) a functional mutant obtained by deleting Q and L at positions 82-83 in the sequence shown in SEQ ID NO: 102, or deleting EQLI at positions 81-84 in the sequence shown in SEQ ID NO: 102, and the functional mutant enables the protein always expressed on the cell membrane in a membrane-bound form and reduces the expression of the secreted form;
(3) the sequence shown in SEQ ID NO: 32, SEQ ID NO: 33 or the sequence shown in SEQ ID NO: 103, SEQ ID NO: 104;
(4) intracellular region of CD258.

12. Genetically modified immune cells, wherein the genetic modification is such that the expression level of one or more of SIRT1 or the functional mutants thereof, SIRT2 or the functional mutants thereof, SIRT3 or the functional mutants thereof, SIRT4 or the functional mutants thereof, SIRT5 or the functional mutant thereof, SIRT6 or the functional mutant thereof, SIRT7 protein or the functional mutant thereof is increased in the immune cells, or the genetic modification is such that the expression level of the CD258 protein or one or more of SIRT1 or the functional mutants thereof, SIRT2 or the functional mutants thereof, SIRT3 or the functional mutants thereof, SIRT4 or the functional mutants thereof, SIRT5 or the functional mutant thereof, SIRT6 or the functional mutant thereof, SIRT7 protein or the functional mutant thereof is increased in the immune cells.

13. The genetically modified immune cell of claim 12, wherein the up-regulation of expression is achieved by:
(1) stimulating the immune cells themselves to increase the expression of one or more of SIRT1 or the functional mutant thereof, SIRT2 or the functional mutant thereof, SIRT3 or the functional mutant thereof, SIRT4 or the functional mutant thereof, SIRT5 or the functional mutant thereof, SIRT6 or the functional mutant thereof, SIRT7 protein or the functional mutant thereof by adding an activator to the immune cells, the activator increases the expression of SIRT1 or the functional mutant thereof, SIRT2 or the functional mutant thereof, SIRT3 or the functional mutant thereof, SIRT4 or the functional mutant thereof, SIRT5 or the functional mutant thereof, SIRT6 protein or the functional mutant thereof, SIRT7 protein or the functional mutant thereof, preferably the activator is selected from: SRT2104 (chemical formula C₂₆H₂₄N₆O₂S₂); CAY10602 (chemical formula C₂₂H₁₅FN₄O₂S); OSS-128167 (chemical formula C19H₁₄N₂O₆);
and/or
(2) transfecting the immune cell with the expression vector which encodes the nucleic acid of SIRT1 or the functional mutant thereof, SIRT2 or the functional mutant thereof, SIRT3 or the functional mutant thereof, SIRT4 or the functional mutant thereof, SIRT5 or the functional mutant thereof, SIRT6 or the functional mutants thereof, SIRT7 protein or the functional mutants thereof, CD258 protein or the functional mutants thereof, to increase the amount of the SIRT1 or functional mutants thereof, SIRT2 or the functional mutant thereof, SIRT3 or the functional mutant thereof, SIRT4 or the functional mutant thereof, SIRT5 or the functional mutant thereof, SIRT6 or the functional mutant thereof, SIRT7 protein or the functional mutant thereof, CD258 protein or the functional mutant thereof in the immune cells, preferably, the SIRT1 or its functional mutant, SIRT2 or its functional mutant, SIRT3 or its functional mutant, SIRT4 or its functional mutant, SIRT5 or its functional mutant, SIRT6 or its functional mutant, SIRT7 protein or the functional mutants thereof, and CD258 protein or the functional mutants thereof are in the form of monomers or in the form of conjugates connected through connecting elements.

14. The genetically modified immune cell of claim 13, wherein the vector is selected from: a retroviral vector, a lentiviral vector or a transposon plasmid, preferably the vector further comprises a CAR, more preferably, the CAR is connected to a regulatory unit through a connecting element, and the regulatory unit is selected from one or more of the SIRT1 or the functional mutant thereof, SIRT2 or the functional mutant thereof, SIRT3 or the functional mutant thereof, SIRT4 or the functional mutant thereof, SIRT5 or the functional mutant thereof, SIRT6 or the functional mutant thereof, SIRT7 protein or the functional mutant thereof, CD258 protein or the functional mutant thereof.

15. The genetically modified immune cell of claim 12, further comprising a connecting element, wherein the connecting element is selected from: T2A (preferably the nucleotide sequence as shown in SEQ ID NO: 12 and the amino acid sequence shown in SEQ ID NO:84), P2A (the preferred nucleotide sequence shown in SEQ ID NO: 13 and the amino acid sequence shown in SEQ ID NO:85), F2A, E2A and IRES (the preferred nucleotide sequence shown in SEQ ID NO: 14), preferably, the connecting element includes or consists of the sequences shown in SEQ ID NO: 84, SEQ ID NO: 85 and SEQ ID NO: 14.

16. The genetically modified immune cell of claim 12, wherein the immune cell comprises a fragment selected from:
GPC3-S1, consisting of SEQ ID NO: 1, SEQ ID NO: 2, SEQ ID NO: 7, SEQ ID NO: 8, SEQ ID NO: 10, SEQ ID NO: 11, SEQ ID NO: 12, and SEQ ID NO: 15 which are spliced and synthesized in sequence,
GPC3-S2, consisting of SEQ ID NO: 1, SEQ ID NO: 2, SEQ ID NO: 7, SEQ ID NO: 8, SEQ ID NO: 10, SEQ ID NO: 11, SEQ ID NO: 12, and SEQ ID NO: 16 which are spliced and synthesized in sequence,
GPC3-S3, consisting of SEQ ID NO: 1, SEQ ID NO: 2, SEQ ID NO: 7, SEQ ID NO: 8, SEQ ID NO: 10, SEQ ID NO: 11, SEQ ID NO: 12, and SEQ ID NO: 17 which are spliced and synthesized in sequence,
GPC3-S6, consisting of SEQ ID NO: 1, SEQ ID NO: 2, SEQ ID NO: 7, SEQ ID NO: 8, SEQ ID NO: 10, SEQ ID NO: 11, SEQ ID NO: 12, and SEQ ID NO: 20 which are spliced and synthesized in sequence,
GPC3-S1A, consisting of SEQ ID NO: 1, SEQ ID NO: 2, SEQ ID NO: 7, SEQ ID NO: 8, SEQ ID NO: 10, SEQ ID NO: 11, SEQ ID NO: 12, and SEQ ID NO: 22 which are spliced and synthesized in sequence,
GPC3-S1B, consisting of SEQ ID NO: 1, SEQ ID NO: 2, SEQ ID NO: 7, SEQ ID NO: 8, SEQ ID NO: 10, SEQ ID NO: 11, SEQ ID NO: 12, and SEQ ID NO: 23 which are spliced and synthesized in sequence,
GPC3-S1B1, consisting of SEQ ID NO: 1, SEQ ID NO: 2, SEQ ID NO: 7, SEQ ID NO: 8, SEQ ID NO: 10, SEQ ID NO: 11, SEQ ID NO: 12, and SEQ ID NO: 24 which are spliced and synthesized in sequence,
GPC3-S1C, consisting of SEQ ID NO: 1, SEQ ID NO: 2, SEQ ID NO: 7, SEQ ID NO: 8, SEQ ID NO: 10, SEQ ID NO: 11, SEQ ID NO: 12, and SEQ ID NO: 25 which are spliced and synthesized in sequence,
GPC3-S1C1, consisting of SEQ ID NO: 1, SEQ ID NO: 2, SEQ ID NO: 7, SEQ ID NO: 8, SEQ ID NO: 10, SEQ ID NO: 11, SEQ ID NO: 12, and SEQ ID NO: 26 which are spliced and synthesized in sequence,
GPC3-S1D, consisting of SEQ ID NO: 1, SEQ ID NO: 2, SEQ ID NO: 7, SEQ ID NO: 8, SEQ ID NO: 10, SEQ ID NO: 11, SEQ ID NO: 12, and SEQ ID NO: 27 which are spliced and synthesized in sequence,
GPC3-S1D1, consisting of SEQ ID NO: 1, SEQ ID NO: 2, SEQ ID NO: 7, SEQ ID NO: 8, SEQ ID NO: 10, SEQ ID NO: 11, SEQ ID NO: 12, and SEQ ID NO: 28 which are spliced and synthesized in sequence,
GPC3-S1E, consisting of SEQ ID NO: 1, SEQ ID NO: 2, SEQ ID NO: 7, SEQ ID NO: 8, SEQ ID NO: 10, SEQ ID NO: 11, SEQ ID NO: 12, and SEQ ID NO: 29 which are spliced and synthesized in sequence,
GPC3-S1E1, consisting of SEQ ID NO: 1, SEQ ID NO: 2, SEQ ID NO: 7, SEQ ID NO: 8, SEQ ID NO: 10, SEQ ID NO: 11, SEQ ID NO: 12, and SEQ ID NO: 30 which are spliced and synthesized in sequence,
GPC3-S1-S3, consisting of SEQ ID NO: 1, SEQ ID NO: 2, SEQ ID NO: 7, SEQ ID NO: 8, SEQ ID NO: 10, SEQ ID NO: 11, SEQ ID NO: 12, SEQ ID NO: 15, SEQ ID NO: 13, and SEQ ID NO: 17 which are spliced and synthesized in sequence.
GPC3-S1A-S3, consisting of SEQ ID NO: 1, SEQ ID NO: 2, SEQ ID NO: 7, SEQ ID NO: 8, SEQ ID NO: 10, SEQ ID NO: 11, SEQ ID NO: 12, SEQ ID NO: 22, SEQ ID NO: 13, and SEQ ID NO: 17 which are spliced and synthesized in sequence.
GPC3-C8, consisting of SEQ ID NO: 1, SEQ ID NO: 2, SEQ ID NO: 7, SEQ ID NO: 8, SEQ ID NO: 10, SEQ ID NO: 11, SEQ ID NO: 12, and SEQ ID NO: 31 which are spliced and synthesized in sequence,
GPC3-C8A consists of SEQ ID NO: 1, SEQ ID NO: 2, SEQ ID NO: 7, SEQ ID NO: 8, SEQ ID NO: 10, SEQ ID NO: 11, SEQ ID NO: 12, and SEQ ID NO: 32 which are spliced and synthesized in sequence,
GPC3-C8B, consisting of SEQ ID NO: 1, SEQ ID NO: 2, SEQ ID NO: 7, SEQ ID NO: 8, SEQ ID NO: 10, SEQ ID NO: 11, SEQ ID NO: 12, and SEQ ID NO: 33 which are spliced and synthesized in sequence,
GPC3-S1A-C8A, consisting of SEQ ID NO: 1, SEQ ID NO: 2, SEQ ID NO: 7, SEQ ID NO: 8, SEQ ID NO: 10, SEQ ID NO: 11, SEQ ID NO: 12, SEQ ID NO: 22, SEQ ID NO: 13, and SEQ ID NO: 32 which are spliced and synthesized in sequence.
CD19-S1A, consisting of SEQ ID NO: 1, SEQ ID NO: 3, SEQ ID NO: 7, SEQ ID NO: 8, SEQ ID NO: 10, SEQ ID NO: 11, SEQ ID NO: 12, and SEQ ID NO: 22 which are spliced and synthesized in sequence,
CD19-C8A, consisting of SEQ ID NO: 1, SEQ ID NO: 3, SEQ ID NO: 7, SEQ ID NO: 8, SEQ ID NO: 10, SEQ ID NO: 11, SEQ ID NO: 12, and SEQ ID NO: 32 which are spliced and synthesized in sequence,
CD19-S1A-C8A, consisting of SEQ ID NO: 1, SEQ ID NO: 3, SEQ ID NO: 7, SEQ ID NO: 8, SEQ ID NO: 10, SEQ ID NO: 11, SEQ ID NO: 12, SEQ ID NO: 22, SEQ ID NO: 13, and SEQ ID NO: 32 which are spliced and synthesized in sequence.
MSLN-S1A, consisting of SEQ ID NO: 1, SEQ ID NO: 5, SEQ ID NO: 7, SEQ ID NO: 8, SEQ ID NO: 10, SEQ ID NO: 11, SEQ ID NO: 12, and SEQ ID NO: 22 which are spliced and synthesized in sequence,
MSLN-C8, consisting of SEQ ID NO: 1, SEQ ID NO: 5, SEQ ID NO: 7, SEQ ID NO: 8, SEQ ID NO: 10, SEQ ID NO: 11, SEQ ID NO: 12, and SEQ ID NO: 31 which are spliced and synthesized in sequence.
MSLN-C8A, consisting of SEQ ID NO: 1, SEQ ID NO: 5, SEQ ID NO: 7, SEQ ID NO: 8, SEQ ID NO: 10, SEQ ID NO: 11, SEQ ID NO: 12, and SEQ ID NO: 32 which are spliced and synthesized in sequence,
MSLN-C8B, consisting of SEQ ID NO: 1, SEQ ID NO: 5, SEQ ID NO: 7, SEQ ID NO: 8, SEQ ID NO: 10, SEQ ID NO: 11, SEQ ID NO: 12, and SEQ ID NO: 33 which are spliced and synthesized in sequence, and
MSLN-S1A-C8A, consisting of SEQ ID NO: 1, SEQ ID NO: 5, SEQ ID NO: 7, SEQ ID NO: 8, SEQ ID NO: 10, SEQ ID NO: 11, SEQ ID NO: 12, SEQ ID NO: 22, SEQ ID NO: 13, and SEQ ID NO: 32 which were spliced and synthesized in sequence.

17. A composition comprising the genetically modified immune cells of claim 12, optionally, the composition further comprises a pharmaceutically acceptable carrier.

18. A method of improving an efficacy of drugs in preventing and/or treating tumors comprising the method according to claim 10 11 mutant, or its functional mutants in the preparation of reagents that improve the efficacy of drugs in preventing and/or treating tumors, preferably, the reagents are genetically modified immune cells (preferably CAR-T cells).

19. A method of treating and/or preventing tumors comprising the step of using the genetically modified immune cells according to claim 12, wherein the tumor is selected from the group consisting of liver cancer, lung cancer, leukemia and mesothelioma, preferably, the genetically modified immune cells are selected from lymphocytes, preferably, the genetically modified immune cells express chimeric antigen receptors (CAR).

20. A method of improving an efficacy of drugs in preventing and/or treating tumors comprising the method according to claim 11 in the preparation of reagents that improve the efficacy of drugs in preventing and/or treating tumors, preferably, the reagents are genetically modified immune cells (preferably CAR-T cells).
